(19) United States
(12) Patent Application Publication
(10) Pub. No.: US 2022/0161047 A1
(43) Pub. Date: May 26, 2022

(54) SYSTEMS AND METHODS FOR PHOTOBIOMODULATION

(71) Applicant: RayBalance, Inc., San Diego, CA (US)

(72) Inventors: Darrel D. Drinan, San Diego, CA (US); William D. Goodrich, Encinitas, CA (US)

(73) Assignee: RayBalance, Inc., San Diego, CA (US)

(21) Appl. No.: 17/407,299

(22) Filed: Aug. 20, 2021

Related U.S. Application Data
(60) Provisional application No. 63/117,423, filed on Nov. 23, 2020.

(51) Int. Cl. A61N 5/06; A61N 5/067

(52) U.S. Cl. CPC A61N 5/0603; A61N 5/0613; A61N 5/067; A61N 2005/0651; A61N 2005/0626; A61N 2005/0632; A61N 2005/0605

(57) ABSTRACT

The present application is directed to systems, devices, and methods for diagnosing, preventing, and treating diseases and disorders through photobiomodulation therapy, either alone or in combination with one or more other therapies. More particularly, the present invention provides photon source devices configured to deliver light to a portion of an organism, which causes a physiological response within that light exposed organism. The invention also provides a system which includes one or more photon source devices and functionality for diagnosing or assessing a disease or disorder, and for monitoring responsiveness of the disease or disorder to treatment with the therapeutic light. Additionally, this application is directed to utilizing the present systems and devices in combination with known adjunctive therapies including devices, services, drugs, biologics, genetics and supplements to produce synergistic optimal therapeutic outcomes.

### FIELD OF THE INVENTION

This application relates to systems, devices, and methods for diagnosing, preventing, and treating diseases and disorders through photobiomodulation therapy, either alone or in combination with one or more other therapies. More particularly, the present invention provides photon source devices configured to deliver light to a portion of an organism, which causes a physiological response within that light exposed organism. The invention also provides a system which includes one or more photon source devices and functionality for diagnosing and/or assessing a disease and/or disorder, and for monitoring responsiveness of the disease or disorder to treatment with the therapeutic light. Additionally, this application is directed to utilizing the present systems and devices in combination with known adjunctive therapies including devices, services, drugs, biologics, genetics and supplements to produce synergistic optimal therapeutic outcomes.

### BACKGROUND OF THE INVENTION

Many diseases and disorders have one or more specific areas of the body which are affected. These areas of the body may require localized therapy, either alone or in combination with systemic therapy, to manage, treat, or cure the underlying disease or disorder, or to manage one or more symptoms thereof. For example, a localized cancer treatment, such as surgery or radiation therapy, may be administered alone, or may be combined with a systemic cancer treatment, such as chemotherapy or another pharmacological therapy. Similarly, if a person experiences a minor musculoskeletal injury, options for localized therapy may include resting, icing, compressing, and elevating the injured area. The localized therapy may be administered alone or may be combined with a systemic therapy, such as an anti-inflammatory agent. In each case, localized therapy may be a first choice for the patient if the probability of successful treatment is relatively high.

If localized therapy is the primary treatment for a particular condition, then systemic therapy may be considered adjunctive. This is because, contrary to localized therapies, systemic therapies expose a greater amount of off-target body mass to the therapy. Different organs, tissue types, and cell types have different interactions with a given systemic therapy, and this complexity increases the probability of undesired side effects and increases the risk-to-benefit ratio of the therapy. Historically, researchers have attempted to lower the risk-to-benefit ratio of systemic therapeutics by increasing specificity and decreasing off-target interactions. These efforts have resulted in the development of biotherapeutics, but even this type of systemic therapy is increasingly recognized as having undesired side effects. In addition, the complexity of the disease or disorder itself often requires a multi-pronged approach, and stimulation or inhibition of a single biochemical pathway may not be sufficient for effective treatment. There is a need for improved, multi-mechanistic, localized therapies which may be combined with systemic therapeutics when necessary. One type of localized therapy which has gained attention in recent years is photobiomodulation therapy (PBMT), also known as low-level light therapy or low-level laser therapy (LLLT).

PBMT utilizes light in various wavelengths to stimulate or inhibit a physiological response, such as repair of tissues by activating biochemical pathways that generate cellular energy. PBMT has been applied for the treatment of hair loss and inflammatory joint diseases due to its ability to reduce inflammation, promote wound healing, and regenerate the hair follicles. However, while some conditions may respond to PBMT, it is not known whether PBMT may be capable of treating additional conditions. The differences between etiologies of different diseases and disorders do not seem to suggest that PBMT would necessarily be beneficial in each case.

One condition for which it is not fully elucidated whether PBMT would be effective for treatment is sensorineural hearing loss (SNHL). SNHL accounts for approximately 90% of all hearing loss. One of the major physiological causes of SNHL associated with age, ototoxicity, infections, or acoustic trauma is the loss of auditory hair and hair support cells within the inner ear. Auditory hair and supporting cells undergo cell death through various mechanisms that include apoptosis, necrosis, reactive oxygen species (ROS) triggered pathways, activation of pro- inflammatory cytokines and chemokines, and modulation of adenosine-mediated signaling pathways. While limited research has investigated the possibility of restoring the auditory hair and supporting cells within the cochlea through molecular and gene-based approaches, to date, PBMT has not been fully clinically validated to mitigate auditory hair and supporting cell loss, and attempts to directly restore hair and supporting cells through regenerative means have had limited success so far.

The physiology of Sensorineural Hearing Loss

Hearing loss affects more than 466 million people worldwide, making it the fourth leading cause of disability globally as of 2018, according to the World Health Organization. Hearing loss is disabling when it occurs at greater than 40 dB in adults and greater than 30 dB in children. WHO estimates that about 15% of all adults globally have some level of hearing loss, while one-third of all adults above the age of 65 have disabling hearing loss. Geographical trends have also been observed with the incidence of hearing loss. Disabling hearing loss in adults is the greatest in Central or Eastern Europe and Central Asia, while the prevalence of disabling hearing loss in children is the highest in sub-Saharan Africa, South Asia and the Asia Pacific. This unequal distribution of hearing loss across different areas of the world reflects the varying lifestyle factors in these regions. In the United States, approximately one in four adults have some amount of measurable hearing loss due to noise exposure.

Sensorineural hearing (SNHL) loss accounts for 90% of all hearing loss and is caused by problems within the inner ear. The degree of SNHL can range from mild to profound. Mild loss of hearing occurs between 26 to 40 dB range, moderate loss occurs in the 41 to 55 dB range, moderately severe loss occurs in the 56 to 70 dB range, severe loss occurs in the 56 to 70 dB range and profound loss is above 90 dB. SNHL can be caused by various factors such as age (presbycusis), ototoxic drugs, acoustic trauma, hereditary diseases, autoimmune diseases of the inner ear, viral or bacterial infections, or Meniere's disease.

Structural Basis of Hearing Loss

The ear is divided into three main parts: the outer ear, middle ear, and inner ear. The inner ear houses the vestibular organ that controls balance, and the cochlear organ that functions in hearing. The cochlea contains three fluid-filled compartments named the scala vestibuli, scala media, and scala tympani. Extracellular fluid, or perilymph, includes the fluid in the scala vestibuli and scala tympani, while the intracellular fluid, or endolymph, is contained within the scala media (also called the cochlear duct). Homeostasis of the endolymph is crucial for sensory transduction through maintenance of endocochlear potential. The scala media contacts the spiral ligaments and the stria vascularis on its lateral wall. These two structural components of the inner ear are also actively involved in normal functioning of the cochlea. The organ of Corti that rests on the Basilar membrane is the sensory organ of hearing. The sensory epithelium of the organ of Corti contains specialized auditory hair cells surrounded by supporting cells. The basilar membrane registers high frequency sounds at its base and low frequency sounds at its apex. It is the movement of the basilar membrane that allows sensory transduction in the inner ear to take place through the function of auditory hair cells. Collectively, these structural units are important for hearing.

In SNHL, either the cochlea or spiral ganglion structures of the inner ear are dysfunctional, leading to loss of hearing. This type of hearing loss can have either i.) sensory or ii.) neural origins. Sensory hearing loss can occur by damage in the organ of Corti that houses auditory hair cells, or by damage to the strial vascularis that normally supports the organ of Corti through generation of endocochlear potential required for sensory hair cells to process sound waves. Neurons of the spiral ganglion that project to the auditory system within the brain are connected to cochlear hair cells. In neural hearing loss, the spiral ganglion or other auditory components are dysfunctional.

Of these two major causes for SNHL, death of the sensory hair cells within the cochlea, which can further lead to degeneration of the spiral ganglion, is the most common. Unlike other species, the mammalian cochlear hair cells are fully developed at the early embryo developmental stage and cannot regenerate themselves in adulthood. Because of this, SNHL is permanent and a link between age-related hearing loss and SNHL is evident. In the United States, an estimated 50% of all adults between the age of 60 to 69, and 80% of adults who are over the age of 85 have hearing loss to the extent that it interferes with their ability to communicate properly on a regular basis. A recent systematic review of the prevalence of age-related hearing loss within Europe found that 30% of men and 20% of women have hearing loss of 30 dB or more by the time they reach age 70, and the incidence increases to 55% of men and 45% of women at age 80. Age-related hearing loss is characterized by hearing loss at higher sound frequencies above 2000 Hz. In children, genetic causes account for more than 50% of hearing loss. Most hearing loss that occurs in the neonatal stage has genetic causes, whereas hearing loss that occurs in adolescents is usually acquired.

The Role of Auditory Hair Cells in Hearing

Hearing is facilitated through electromechanical transduction in which hair cells of the cochlea play a crucial role in detection of stimuli that is converted into neural impulses and transmitted to the brain. Auditory hair cells convert sounds waves into electrical impulses, which are transmitted to the auditory system within the brain through a process that converts the mechanical energy of sound into electrical energy. Stereocilia that are present on hair cells generate these electrical impulses through their movement in response to sound waves. The movement of stereocilia activates ion channels while they move, creating action potential from the potassium ions present in endolymph. Additionally, calcium ions are also responsible for some part of electrical impulse generation, although its relative concentration within the endolymph is lower than potassium ions. The influx of K+ and Ca2+ ions results in receptor potential that can open voltage gated calcium channels, which release neurotransmitters that trigger the action potential.

There are two types of hair cells within the basilar membrane of the organ of Corti in the inner ear. The outer hair cells (OHC) are arranged in three rows, and the inner hair cells (IHC) are arranged in one row. Although the 12,000 OHCs outnumber the approximately 3,500 IHCs within the cochlea, the IHCs have much denser innervation and are the major sensory receptors that enable hearing through afferent projection to the brain. Conversely, the OHCs are rich in efferent projections on their terminal ends that come from the auditory system in the brain.

For a long time, the role of OHCs in facilitating hearing was unclear, but it is now known that the OHCs act as a ‘cochlear amplifier’ that augments the sensitivity and frequency of hearing. These OHCs move in response to electrical signals generated from sound waves, and the resulting mechanotransduction is due to a reverse transduction process that creates energy within the cochlea. Another important aspect of how sound is transduced within the cochlea is how different vibration frequencies are distributed within the cochlea. Higher sound frequencies displace the basal end of the cochlear duct, while lower frequencies produce maximal displacement in the apical end of the basilar membrane within the cochlear duct. Disruptions to this process can result in various degrees of damage to hearing, or even complete hearing loss. When hair cells are damaged at the basal end of the cochlea, it causes high frequency hearing loss, whereas damage to hair cells at the apical end of the cochlea causes low frequency hearing loss.

Auditory hair cells are subject to various sources of stress, and thus loss, that can be caused by exposure to exogenous chemicals, environmental and occupational factors or genetic causes. The major non-genetic causes of auditory hair cell loss that contribute to hearing impairment include age-related degeneration, ototoxicity from therapeutic drugs or exogenous chemical exposure, acoustic trauma from noise exposure and infections. The underlying cellular mechanisms of auditory hair cell death due to each of these causes vary. Understanding the physiology of hair cell loss can support the development of new treatment methods for its prevention or restoration. Indeed, there are several registered clinical trials with the US National Institute of Health that mostly target prevention of the cell death pathways of auditory hair cells.

Activation of specific cell death pathways, pro-inflammatory molecules and pro-cell death proteins within auditory hair cells has been found to occur in response to certain triggers of hearing loss. Previous studies have highlighted molecular signatures associated with auditory hair cell death in age-related hearing loss, acoustic trauma, response to ototoxic drugs and infections. The sequence of cellular events that occur leading to auditory hair cell death and ultimately loss of hearing is presented.

Cellular Mechanisms of Auditory Hair Cell Loss

Death of auditory hair cells can occur due to various triggers such ototoxic therapeutic drugs that mainly include the aminoglycoside antibiotics, platinum-based chemotherapeutic drugs like cisplatin, viral infections, hypoxia within the cochlea, noise exposure, electrode insertion trauma, and infections such as meningitis. After an event that causes injury to the cochlea, a common sequence of molecular events occurs in which signaling cascades are initiated to promote inflammation, cell death and cell survival of auditory hair cells. The fate of the hair cell is a result of extensive crosstalk between these multiple pathways. While the exact role of cell survival pathways that are initiated after a cochlear insult are less understood, the steps underlying activation of cell death and pro-inflammatory pathways are outlined. In brief, auditory hair cell loss mechanisms can occur through apoptosis, pro-inflammatory cytokines, reactive oxygen species, and potentially through adenosine mediated signaling. Additionally, different modes of apoptosis may be initiated depending on whether the trigger is age-related, due to acoustic trauma, or mediated by ototoxicity from therapeutic drugs or exogenous chemical exposure.

Apoptosis of Auditory Hair Cells

The major pathway of auditory hair cell death following a stress signal is through the intrinsic apoptosis pathway that is executed in the outer membrane of the mitochondria. The Bcl2 family members are the central proteins of the intrinsic apoptotic pathway and the major hallmark of activation of this pathway is upregulation of Bcl2 like-protein 4 (Bax), followed by downregulation of Bcl2. In turn, pro-death proteins, such as cytochrome C, are released into the cytosol through the formation of mitochondrial outer membrane permeabilization (MOMP). The ‘apoptosome’ is formed by binding of cytochrome C and Apaf-1 together, which is responsible for leading the cell into caspase-dependent or caspase-independent cell death. Conversely, the extrinsic apoptosis pathway is primarily initiated by the tumor necrosis factor (TNF) family members that transmit death signals across the cell membrane and activate the execution phase of apoptosis through caspase 8. While apoptosis is the primary mechanism of cell death of auditory hair cells in response to stress signals, some level of necrosis, or chaotic cell death, does also occur. Prolonged activation of the signaling molecule, JNK, can switch the hair cell from apoptosis to necrosis, and this occurs in hair cell loss in response to ototoxic drug exposure, acoustic trauma and TNF-alpha initiated cell death of hair cells. Inhibiting JNK directly protects hair cells from death and results in protection from hearing loss.

Hair cell death (apoptotic) mechanisms of age-related hearing loss [0059] Age-related hearing loss is associated with increased expression of Bax and decreased Bcl2 expression within the cochlea. Decreased expression of Bcl2 allows p53 that normally binds to Bcl2 in the mitochondria to be released, allowing p53-mediated transcriptional activation of proapoptotic genes. A recent study analyzing the expression of genes in OHCs and IHCs by microarray analysis found that 83% of deafness-related genes are expressed in auditory hair cells. Comparison of gene expression in IHCs versus OHCs identified Bcl2 as one of the top ten differentially expressed genes between the two types of hair cells. Both Bcl2 and Bcl6 have increased expression in IHCs versus OHCs. According to the authors, this could explain why OHCs are observed to be more susceptible to early cell death compared to IHCs.

Another recent study revealed that age-related hearing loss is caused by damage to sensory cells in the inner ear, in contrast to the generally accepted notion based on previous studies that implicated the stria vascularis instead. Prior to this study, the concept that age-related hearing loss has metabolic causes was due to the correlation of high frequency hearing loss with strial degeneration in animal models, among which the aging gerbil has provided much reliable data.

One research group examined hair cells, strial tissues, and auditory nerve fibers in 120 post-autopsy human inner ears. Notably, the authors found that the extent of hearing loss correlated well with the amount of hair cell loss. Previously, the stria vascularis was considered to be the ‘battery’ that powers the inner ear. In this study, although strial degeneration was observed throughout the cochlea, statistical modeling showed that a large proportion of hair cell death had already taken place by the time strial atrophy occurred. Loss of OHCs observed in aged human ears would also make the cochlear amplifier dysfunctional, indicating that OHC death occurs before strial degeneration and is functionally important. Interestingly, age-related hair cell death was found distributed throughout the cochlea, but the death of IHCs was greater in the basal half of the cochlea (pertaining to high-frequency) than the apical half (pertaining to low-frequency). Additionally, the amount of OHC survival was an accurate predictor for thresholds, while IHC survival was less important for threshold prediction.

This study also showed that auditory hair cell loss follows a different pattern in humans compared to aging animal models, likely due to chronic acoustic trauma that humans are exposed to throughout their life in urban and industrial areas. The amount of OHC and IHC loss that was observed in the basal half of the cochlea in human ears has not been observed in aging animal models, suggesting that loss of hair cells in this region is due to noise exposure. It also indicates that hair loss occurring in the apical cochlear region is likely due to age and less affected by acoustic trauma.

Hair Cell Death Mechanisms of Ototoxicity

The aminoglycoside antibiotics (gentamicin, kanamycin, amikacin and neomycin) produce ototoxicity by being transported into cochlear hair cells and supporting cells through mechanisms that include endocytosis and mechanotransduction. Gentamycin is transported into basal hair cells, and these hair cells also have lower antioxidant expression making them more vulnerable to the presence of ROS mediated damage. Collectively, ototoxicity appears to affect OHCs more than IHCs with more damage within the basal turn of hair cells.

Cell death due to ototoxicity occurs through the intrinsic apoptotic pathway by the activation of Bax, subsequent release of cytochrome C from the mitochondria, and activation of caspase-3 leading to DNA degradation. Cisplatin, a platinum-based drug, also induces the production of free radicals within the cochlea. Evidence of necrosis due to cisplatin ototoxicity is also apparent, however it has not been well characterized.

Hair Cell Death Mechanism of Acoustic Trauma

Hair cell death resulting from acoustic trauma is the most understood out of all the triggers of hearing loss. Loud sounds can displace large portions of the tympanic membrane sending large mechanical waves into the inner ear. In turn, these waves of mechanical energy rapidly displace cochlear inner ear fluid, which causes shearing force damage to the inner ear. Evidence suggests that acoustic trauma restricts blood flow into the cochlea causing hypoxic conditions that injures auditory hair cells. Due to hypoxia, marginal cells of the stria vascularis release reactive oxygen species causing further damage, although the mechanism of how this happens is not clear.

Acoustic trauma-induced cell death of auditory hair cells can occur through a combination of the intrinsic apoptosis pathway, extrinsic pathway, and regulated necrosis. The cytokine, TNF-α, is released within the cochlea following acoustic trauma. TNF-α binds to receptors TNFR1, TRADD and FADD that activates the extrinsic apoptotic pathway through recruitment of caspase-8. The intrinsic apoptotic pathway is activated through TNF-α-mediated upregulation of p38 and MAPK signaling pathways in the inner ear sensory epithelium that promotes Bax expression and the subsequent release of cytochrome C from the mitochondria. Oxidative stress also activates the intrinsic apoptotic pathway in hair cells through caspase-3 dependent cell death. Regulated necrosis occurs through the RIPK3/RIPK1 pathway or JNK pathway that is also active in apoptosis, but when activated for a prolonged period of time, it initiates necrotic cell death mechanisms. Previous studies have shown that inhibiting apoptotic caspases upregulates necrosis proteins RIP1 and RIP3, and vice versa. Additionally, employing a necrosis inhibitor reversed necrotic death of hair cells in rats.

Both JNK and p38 are pro-apoptotic pathways. The protein p38 upregulates Bax, while JNK can activate apoptosis either through phosphorylation of proteins required for mitochondrial cell death, or through translocation to the nucleus to promote the expression of other pro-apoptotic proteins like TNF-α, FasL, Bak, Bim, and Bax through phosphorylation of p53 and c-Jun. Interestingly, NF-κb activation also attempts to upregulate Bcl2 expression and Bcl-xl to rescue auditory hair cells from apoptosis. Significant crosstalk occurs between these different signaling pathways. When the balance between pro-death and pro-survival pathways favors apoptosis, auditory hair cell death occurs.

Reactive Oxygen Species (ROS) Mediated Cell Death

A key event after exposure to aminoglycoside antibiotics (infection), cisplatin, acoustic trauma, or electrode insertion trauma, is increased levels of ROS in the inner ear, which contributes to the death of auditory hair cells, ultimately leading to hearing loss. ROS are free radicals containing oxygen that are produced by neutrophils, monocytes, and macrophages. Within the cell, ROS are generated within the mitochondria, and these super reactive molecules can cause significant hair cell death in age-related hearing loss. This is thought to be due to inefficient blood flow or environmental factors that can lead to damage of the mitochondrial membrane and DNA. The generation of ROS is dependent on the presence of superoxide anions (O2-), which are either produced enzymatically from NAD phosphate oxidases (NADPH) on phagocyte membranes, or as a by-product of the electron transport chain (ETS) that produces ATP within the mitochondria. High levels of ROS from its overproduction can initiate cell death through apoptotic pathways. Although there are protective mechanisms within the cell to neutralize the effects of oxidative stress, such as through antioxidant enzymes like superoxide dismutase and glutathione peroxidase, the fate of the cell is decided through the balance of ROS levels and antioxidant activity.

Auditory Hair Cell Death through Pro-Inflammatory Cytokines and Chemokines

TNF-α is the major pro-inflammatory cytokine that is released by the stria vascularis and spiral ligament after an ear injury, and the events that are triggered by its release can cause cochlear hair cell death. TNF-α levels are elevated in the cochlea after gentamycin exposure, cisplatin exposure, noise exposure, electrode insertion trauma and autoimmune diseases. Its expression promotes the generation of superoxide free radicals into the cochlea. It also promotes the migration and adhesion of other pro-inflammatory molecules like neutrophils, macrophages, monocytes, lymphocytes, eosinophils and basophils into the injured cochlea through expression of Interleukin 1-beta (IL-1β), MCP-1, MIP-2, siCAM-1, VCAM-1, ICAM-1 and VEGF. In particular, IL-1β expression levels are very high in the cochlea after gentamicin exposure, electrode insertion trauma and in autoimmune ear diseases. TNF-α binds to the TNF receptor 1 (TNFR-1) on the surface of hair cells to initiate cell death signaling pathways. Extrinsic apoptosis is activated through recruitment of caspase 3 and -7, and intrinsic apoptotic pathways are initiated through activation of Bax and truncation of Bid. TNF-α also activates other pro-inflammatory and pro-apoptotic signaling pathways mediated by MAPF, JNK and p38 in auditory hair cells. Thus, after a stress signal, the collective actions of TNF-α promote inflammatory responses and activate apoptosis-mediated cell death pathways in the inner ear.

Adenosine signaling mediated loss of hair cells

Adenosine is an important signaling molecule in the central nervous system. Adenosine is released from cochlear tissues after exposure to stress such as acoustic trauma. It is also generated from extracellular ATP through the activity of ectonucleotidases. There are three high affinity adenosine receptors in the human cochlea (A1, A2, A3). It is widely believed that the balance between A1 and A2 receptors is critical for cochlear response to various stresses. The A1 receptor is involved in protection from inflammation, while A2receptors are pro-inflammatory, and the balance between these two receptors is critical for determining cochlear response to oxidative stress following a stress trigger. Stimulation of the Al receptors has otoprotective effects. After exposure to acoustic trauma, a transient impairment of hearing, called a temporary threshold shift, can occur. When acoustic trauma constantly elevates threshold shifts, a permanent threshold shift (PTS) occurs. Previous studies have shown that activation of Al receptors mediates OHC recovery after exposure to noise, and their activity results in a reduction of PTS. Pre-treatment of cochleas with an adenosine analog (R-PIA) also decreased hearing loss in animal models exposed to 4 kHz octave band noise. Furthermore, activation of A1 receptors with R-PIA also enhanced production of antioxidants superoxide dismutase and glutathione peroxidase that counters the effect of ROS in the cochlea after noise exposure. Additionally, tissue protective effects of adenosine signaling within the cochlea after noise exposure and stress from ototoxic drugs have been demonstrated using drugs that activated the Al adenosine receptors. Overall, adenosine signaling through the A1 receptors improves the blood flow and oxygen supply, increases antioxidant production and counters the effects of ROS in the cochlea to protect the survival of OHCs after acoustic trauma.

Cellular Mechanisms—Regulation of auditory hair cells by the Hippo Signaling Pathway

The Hippo signaling pathway, also known as the Salvador-Warts Hippo pathway, controls the development of organ size by regulating cellular proliferation and apoptosis through a cascade of signaling events that are tissue-specific. In the development of the inner ear, the Hippo pathway and its downstream effector proteins, the Yes associated protein (YAP)/Tead pathway, function in a precisely timed manner to control the amount of proliferation that occurs in the development of the inner ear. The YAP/Tead pathway activates proliferation and anti-apoptotic genes, making its overall effect pro-survival, and the Hippo pathway normally represses their pro-survival functions to prevent reactivation of cellular proliferation and growth. Research has shown that the activation of YAP is important for differentiation of hair cells in a zebrafish model. A recent study demonstrated that after loss of hair cells, reactivation of the YAP/Tead pathway could restore proliferation in mammalian cochlea. This study suggests that inhibition of the Hippo pathway along with activating YAP in the inner ear could drive restoration of hair cells through stimulating a proliferative response in supporting cells of the inner ear.

Other physiological problems within the inner ear can also lead to hearing loss. Loss of auditory hair cells in the cochlea may contribute to the development of SNHL as a consequence of these ear disorders.

Tinnitus is the perception of sound in the ear or head without any external acoustic stimulus. The condition affects more than 50 million people in the United States and 70 million people in Europe. Primary tinnitus can lead to SNHL. Damage to the stereocilia of the outer hair cells can act as a pathophysiological trigger for acute tinnitus. Additionally, tinnitus is one of the earliest symptoms of age-related SNHL. Treatment methods for tinnitus have been severely limited due to a lack of understanding of how exactly tinnitus occurs. Current treatment methods use prescription drugs such as sedatives, antidepressants, local anesthetics and antihistamines, or other methods like Tinnitus Retraining Therapy, repetitive Transcranial Magnetic Stimulation (rTMS), antioxidant therapy, or sound therapy.

Previous studies have suggested that peripheral tinnitus may arise from OHC dysfunction within the cochlea. Damage to OHCs can cause changes in the endocochlear potential, leading to unprompted cochlear activity. This lends support to the connection between the development of tinnitus and acoustic trauma, as OHCs are the first cells that are damaged within the ear after this type of ear trauma. Death of OHCs and IHCs have been observed in rodent models of tinnitus, but it has not been well studied in humans. Additionally, the N-methyl-D-aspartate (NMDA) receptor that resides in IHCs has been implicated in noise-induced tinnitus. Recent evidence has shown that blocking the activation of NDMA receptors prevents IHC loss after acoustic trauma.

Otitis media is a very common ear infection that affects approximately 700 million people worldwide. Otitis media is initiated by a viral upper respiratory infection involving mucosa of the nose, nasopharynx, middle ear mucosa and Eustachian tubes that leads to colonization of bacterial and viral organisms within the middle ear, eventually causing fluid buildup. The majority of cases of otitis media are in children. In the United States, 70% of all children experience at least one case of acute otitis media by their second birthday. Acute otitis media can develop into chronic suppurative otitis media and more than 50% of people with this condition develop hearing loss.

Previous research has supported the role of auditory hair cell loss in otitis media. Auditory hair loss of OHCs and IHCs has been previously reported in animal models with otitis media. Furthermore, histopathological examination of 614 temporal bones with otitis media, including chronic and purulent otitis media, found significant loss of OHCs and IHCs as well as a decrease in the area of the stria vascularis. These studies indicate that auditory hair cell loss can occur as a consequence of otitis media.

Tympanic membrane (TM) perforation is the rupturing of the eardrum that occurs as a secondary complication of otitis media or due to trauma. Different pore sizes can occur in TM perforation and the current incidence in the United States is unknown. However, as of 2015, approximately 150,000 tympanoplasties were performed annually. Repair of the eardrum after an acute perforation occurs due to the presence of stem cells and progenitor populations. Newly proliferated keratinocytes are present in the epithelial and mesenchymal layers of the TM at the location of the perforation and surrounding the manubrium. These cells are also present throughout the epidermal membrane even far away from the TM hole, indicating that long distance signaling may occur to repair the TM.

In most cases, blast injury to the ear that causes perforations in the tympanic membrane leads to permanent hearing loss due to irreparable trauma of the cochlea. The pressure of a lethal blast for human cochlea is between 414 and 552 kPa, but an estimated 50% of TM perforations can occur with blast pressures as low as 104 kPa. Studies in mouse models with TM perforations have indicated that hearing loss that occurs after this type of damage is not limited to the intracochlear membrane. Loss of OHCs at the basal turn of the cochlea, reduced spiral ganglion neurons, and reduced afferent nerve synapses were all observed to be a part of the inner ear physiology that leads to permanent hearing loss after a TM perforation.

Balance disruptions—Disorders of the inner ear that cause balance disturbances include symptoms of dizziness, unsteadiness, and a feeling of spinning. Labyrinthitis and Meniere's disease are two disorders that cause balance disruptions and dizziness. Labyrinthitis is an infection or inflammation of the inner ear that affects the vestibular system, which plays a crucial role in maintaining balance. The vestibule is close to the cochlea in the inner ear and vestibular hair cells are crucial ‘balancers’ within this system. Interestingly, unlike auditory hair cells, mammalian vestibular hair cells have some regenerative potential. Meniere's disease is characterized by the feeling of deep pressure inside the ear that leads to tinnitus, vertigo, and loss of balance. Meniere's disease is quite rare and usually affects only one ear, but it can lead to irreversible hearing loss, potentially through repeated damage of auditory hair cells in the inner ear. The disease affects 2 out of 1000 people in the United States, with the majority of people diagnosed with the condition being over the age of 40. An increase in auditory hair cell death has been observed in patients with the disease, reinforcing the theory that hair cell death causes unilateral functional deafness in Meniere's disease. Although vestibular hair cells appear to be less affected than auditory hair cells, their gradual decline over a span of 15 years has also been observed.

Dementia—Dementia is a group of conditions that affects brain function causing memory loss, impaired thinking or problem-solving abilities and problems with language. The condition affects 47 million people worldwide and one in ten people over the age of 65 have Alzheimer's disease in the United States, with prevalence doubling every five years after that. Previous research has investigated the link between age-related decline in sensory systems, including the auditory system, and neurodegenerative diseases like Alzheimer's disease and dementia. Based on this, there are several theories that link an impaired auditory system with cognitive decline. One theory is that reduced auditory stimulation due to SNHL can directly cause degradation of other cognitive processes through changes in brain structure that make it susceptible to the development of dementia. Other theories postulate that more cognitive resources are needed for people with hearing impairment to recognize speech-in-noise, and this makes resources in the medial temporal lobe (MTL), where auditory cognitive processing takes place, unavailable for higher cognitive tasks leading to dementia on its own, or through functional interaction with the pathology of Alzheimer's disease. Another hypothesis is that hearing loss and dementia have a common mechanistic pathology that affects the cochlea, the auditory pathway and the brain cortex that causes dementia. Supporting this theory, abnormal expression of identical proteins that have common downstream targets and pathways have been observed in both dementia and age-related hearing loss. These proteins include vascular endothelial growth factor (VEGF), SIRT1- PGC1α, and CaMKKβ-AMPK. Interestingly, overexpression of these proteins causes dysfunction of auditory hair cells within the cochlea. A transgenic mouse expressing amyloid-β derivatives, which are known drivers of Alzheimer's disease, in cochlear hair cells had early-onset hearing defects that included loss of high frequency sound perception (usually associated with age-related hearing loss), and auditory hair cell loss in the basal region of the cochlea. Overexpression of the protein, tau, another key protein in Alzheimer's disease pathology, in cochlear hair cells also synergistically enhanced hearing impairment in these mice.

PBMT prophylactic prevention of cochlear hair cells and supporting cell loss has been researched demonstrating potential mechanisms through which PBMT may mitigate or prevent hair cells and supporting cell loss. The ability of laser light to regenerate hair growth was demonstrated in the early 1960's by a Hungarian physician, Endre Mester. While investigating whether lasers have carcinogenic potential in animal models, he found that a low-power ruby laser healed wounds more rapidly and improved hair growth of shaved mice. This was the first demonstration of Low-Level Light therapy (LLLT), which is now more commonly called photobiomodulation therapy (PBMT). There are now hundreds of studies in both the clinical setting and in animal models that demonstrate the benefits of PBMT in human disease applications. These include alopecia, joint inflammation, musculoskeletal pain, osteoarthritis, rheumatoid arthritis, depression, acne, several types of cancer including photodynamic therapy for anti-tumor immunity, oral mucositis, pressure and diabetic ulcer wound healing, bone healing, Alzheimer's disease, skin and mucosal infections, rosacea, traumatic brain injury, lung inflammation and autoimmune diseases like thyroiditis, alopecia areata, and psoriasis.

The term PBMT represents the broad capacity of the technique to heal tissues, as its use is not limited to only lasers and can include both coherent and non-coherent sources of light. Both red light and near infrared (NIR) light are most commonly used in treatment methods that use PBMT. Treatment of human tissues with light does not harm living tissues and it offers a wide wavelength range of between 650 to 1000 nm. The general principle of using light within these ranges is that long wavelength light can stimulate cellular metabolism to initiate the healing and reparative effects seen in various applications using PBMT. Hemoglobin and myoglobin, two of the major chromophores in the human body, preferentially absorb photons at wavelengths below 600 nm. This leaves cytochrome c oxidase as the principal chromophore that activates cellular respiration in the mitochondria with light in the NIR wavelength range. Typically, superficial tissue is treated with light in the range of 600 to 700 nm and longer wavelengths in the 780-1000 nm window are used for deeper tissues (>1 cm), as light in this range can penetrate further. Notably, light in the range of 700 to 770 nm has limited ability to stimulate cellular respiration and biochemical activity within tissues.

Initial sources of light used in PBMT were laser-based. Mester used a HeNe laser that emitted light at 632 nm. For years, the application of a laser in PBMT was standard, accounting for more than 85% to 90% of all studies. More recently, light emitting diodes (LED) are being increasingly applied in PBMT due to several advantageous features. LEDs do not produce significant thermal energy, so there is limited potential risk for injury to tissues that undergo treatment. Unlike lasers, LED sources can cover a wider area of treatment compared to lasers as they have a larger bandwidth. As a therapeutic device, LEDs have been given FDA non-significant risk status. Additionally, LEDs are compact and relatively inexpensive, making them cost-effective approaches to application in PBMT.

Importantly, there are various factors that can affect the efficacy of PBMT as a therapeutic approach. These variables include the design of the light source and its associated energy parameters. The dose of PBMT is usually defined as J/cm2 and utilizes these primary inputs: irradiance (power density), fluence (energy density), time of exposure, area of exposure, sequence of illumination and wavelength of light. The fluence employed in applications of PBMT is usually within the range of 0.5 to 20 J/cm2 and treatment of deeper- seated tissues can employ fluences of up to 50 J/cm2. The irradiation parameter also has a wide range from between 1 to 250 mW/cm2 and is highly dependent on the spot size of treatment. There has also been some debate about whether the use of pulsed light or a continuous wave (CW) is more effective. Some studies suggest that using pulsed light at a specific peak power density is safer than using the same power density as CW. Additionally, the frequency of pulses and time of each pulse can also affect the efficacy of therapy. Studies have shown varied results in whether using pulsed light is as or more effective than CW. As a therapy, PBMT is often repeated for a certain number of times per week depending on the condition that it is used to treat. The frequency and time between treatments also affects how well it works. The sequence of light illumination has also shown to be an influence on the physiological response. In summary, variables that strongly affect the efficacy of PBMT include the irradiance of the light source, the area of skin or tissue exposed, the depth of the targeted tissue tissues, time of exposure, illumination sequence, light pulse frequency, and distance from the light source to the skin.

Mechanisms of PBMT in protection and stimulation of cellular growth—The cellular mechanism through which PBMT elicits its effects on healing tissues is both stimulatory and inhibitory at the molecular level. The major clinical applications where PBMT has successfully been applied have common underlying molecular mechanisms that have demonstrated effects to reduce inflammation, promote tissue regeneration and prevent damage or death of cells or tissue due to a disease or injury. This is through altering the redox state of the cell, which further activates downstream intracellular signaling pathways to modulate cell proliferation, survival, and death pathways for an overall healing effect on the treated tissue.

PBMT does not produce thermal heat in cells. Instead, the effects of PBMT are photochemical, in which the light is used to create biochemical changes within the cell to produce energy. This process has been compared to photosynthesis in plants. The effects of using low intensity light (between 650 to 1000 mu wavelength range and 0.5 to 20 J/cm2 energy density) are not damaging to the cell. Similar to the way in which plants activate photosynthesis through chlorophyll present in plant cells, when PBMT is applied to human cells, NIR light activates proteins within the cell that increase mitochondrial cellular respiration. Three main proteins that act as photoacceptors in response to NIR light within mammalian tissues are hemoglobin, myoglobin, and cytochrome C. The exact mechanism of PBMT in regenerating hair and supporting cells has not been fully elucidated, however, the most accepted theory is through cytochrome c oxidase mediated increase of ATP production in the mitochondria, formation of reaction oxygen species and activation of transcription factors that activate downstream proteins that regulate cell proliferation, cell migration, cytokine levels and mediators of inflammation. The following steps are postulated to occur through PBMT application to cells.

Increase in ATP production—Complex IV of the respiratory electron transport chain (ETC), known as cytochrome c oxidase, is the most important component of cellular response to PBMT. Several lines of evidence have indicated that when PBMT is applied to cells, a photon of light is absorbed by a chromophore within the mitochondria. This photon can become excited and pass through the ETC that generates ATP as its final product through a proton gradient that is created as electrons pass through the chain. This ATP is stored as energy that is used for various cellular processes. The most widely accepted theory to date is that cytochrome c acts as an acceptor for an activated photon from PBMT through the electron transport chain. This has been demonstrated in multiple studies that provide experimental evidence for an increase in energy metabolism and ATP-mediated activation of numerous signaling pathways after PBMT application.

Another major observation of the effects of PBMT at the cellular level is the release of nitric oxide (NO) from cells. Normally, cellular respiration is inhibited through replacement of oxygen with NO on cytochrome c oxidase, which decreases ATP production. The exact mechanism by which NO release stimulates an increase in ATP production following PBMT is hypothesized to occur either through dissociation of existing NO from cytochrome c oxidase allowing cellular respiration to occur, or through cytochrome c oxidase mediated reduction of nitrite to produce NO that increases its bioavailability.

Formation of reaction oxygen species—In the last step of the ETC, oxygen is converted to water. Reactive oxygen species (ROS) are a by-product of this process. Since PBMT activates the ETC, oxygen is converted to water and there is a subsequent increase of ROS within the cell that changes its redox state. Transcription factors that are responsive to a change in cellular redox levels are then activated to promote protective cell survival effects such as an increase in cell proliferation and migration. Some of the key transcription factors that are activated include redox factor-1 (Ref-1) dependent activator protein-1 (AP-1), NF-κB, hypoxia-inducible factor (HIF)-1, and factor/cAMP-response element-binding protein (ATF/CREB).

Modulation of immune cells—One of the largest pro-survival cellular effects elicited by PBMT is through immune cell activation. Light at specific wavelengths can trigger degranulation of mast cells that releases the pro-inflammatory cytokine, TNF-α, from cells leading to infiltration of leukocytes into tissues. Additionally, PBMT activates and increases the proliferation of lymphocytes, as well as enhances the phagocytic action of macrophages. Fibroblast and epithelial cell motility, which are important for wound healing, is also improved.

Increased O2 levels—PBMT induces smooth muscles to relax which can cause vasodilation in treated tissues. This effect allows more immune cells to infiltrate into tissues, as well as increases the availability of oxygen in these tissues. Both effects enhance healing in treated tissues so PBMT has been used to successfully treat joint inflammation.

Alteration of apoptosis—Recent studies have reported the ability of PBMT to alter apoptotic pathways within treated tissues. Human fibroblast cells treated with infrared (IR) light altered the balance of anti-apoptotic protein, Bcl2, and pro-apoptotic protein, Bax, by decreasing Bax expression. This directed the cells into survival instead of death. Furthermore, IR-treated cells demonstrated inhibition of UVB-mediated activation of caspase-3 and caspase-9. The modulation of Bcl2/Bax was further shown to be controlled by the p53 signaling pathway, indicating that PBMT may impact this master transcription factor in mediating its tissue protective effects. Other studies demonstrated the ability of PBMT to inhibit apoptosis in response to cytotoxic substances in multiple cell types through upregulation of anti-apoptotic proteins. In this study as well, Bcl2 was upregulated and Bax had decreased expression. Additionally, increased mitochondrial biogenesis as measured by expression of fission and fusion proteins has also been observed in response to light therapy. An increase in mitochondrial biogenesis increased ROS and NO concentration.

Modulation of the mitochondrial interfacial water layer—Another alternative theory for the cellular mechanism elicited by PBMT in increasing ATP levels within the cells has been postulated by Andrei Sommer's group. This group postulates that cytochrome c oxidase is not the primary acceptor of photons from NIR as the most popular theory suggests. Sommers et al. hypothesize that water present within the mitochondria prevails as interfacial water layer (IWL). Through early experimental evidence, they demonstrated that two to three monolayers of nanoscopic IWL can be modulated through PBMT at 670 nm, and this effect was not limited to that wavelength. A decrease in intramitochondrial viscosity with light treatment was observed, which the authors relate to the increase in ATP production. They suggest that ATP synthase, the mitochondrial motor that synthesizes ATP, rotates faster under lower viscosity conditions, producing more ATP when exposed to NIR light. The authors also reason that this mechanism is more likely be relevant for pulsed light at low frequency such as 1 Hz. Additionally, they relate levels of ROS to intramitochondrial viscosity to explain how it impacts ATP production. Previous studies have shown that an increase in ROS in the cell is associated with a concomitant decrease in ATP production. One group hypothesizes that an increase in ROS, often seen in pathological conditions that cause oxidative stress and subsequent cell death, causes a temporary increase intramitochondrial interfacial water layer viscosity, which leads to decreased ATP levels. According to them, reduction of this viscosity through light therapy then restores ATP production.

Review of PBMT applications in hearing loss and inner ear disorders—Generally, PBMT mediates its protective, growth-promoting and regenerative effects through both inhibitory and stimulatory cellular mechanisms, in which biological processes that promote cell death are inhibited and cell survival pathways that promote proliferation and migration of epithelial cells and release of immune cells is activated. The mechanisms described above have been demonstrated in multiple cell types in vitro, in animal models of wound healing and inflammation, and in human clinical studies.

The potential of PBMT in regenerating the hair follicle is well known. This treatment method, using a laser comb, was approved by the FDA for treatment of both male and female pattern hair loss in 2007 and 2011, respectively. Multiple studies of hair regrowth in animal models and in clinical trials have provided promising results using light within the range of 635 to 650 nm. The main mechanism of PBMT stimulated hair growth is postulated to be through epidermal stem cell stimulation in the hair follicle bulge that shifts follicles into its growth phase, termed the anagen phase. This technique is thought to rely on the most crucial cells within the hair follicle in the dermal papilla. Epithelial stem cells that reside in the hair follicle bulge can proliferate and differentiate further downstream in response to signals from the dermal papilla. Therefore, although the concept of using PBMT for stimulating hair growth is not new, approaches using PBMT in the treatment of alopecia and other types of adult-pattern hair loss are dependent on the regeneration potential and growth phase of the hair follicle.

100661 Given the proven efficacy of PBMT in these biological processes, it becomes imperative to investigate the potential of PBMT in restoring hearing in SNHL, or in prevention of hearing loss in incidences wherein it is likely to occur, such as the presence of middle and inner ear disorders such as chronic suppurative otitis media or after an incidence of acoustic trauma. There have been a few studies that examined the potential of PBMT in benefiting hearing loss and even fewer that investigate using PBMT to treat cochlear hair cells. This is due in part to the difficulty in obtaining human samples to conduct studies. However, novel approaches in using PBMT in preventing or treating SNHL may be focused on stimulation of cochlear hair cell growth, prophylactic prevention of auditory hair cell loss, or stimulating repair of other sensory components involved in the pathology of hearing loss. In this section, we focus on a review of studies that support the therapeutic use of PBMT in treating auditory hair cell loss that causes SNHL.

Applications of PBMT in cochlear hair cell protection or regrowth in contrast to non-mammalian vertebrates that contain stem or progenitor cells that have regenerative potential within the inner ear, mature mammalian cochlear hair cells do not have regeneration potential. Hematopoietic stem cells that have bone marrow origin are found within the mature inner ear, but evidence has not supported their development into hair cells. Furthermore, studies conducted with mouse cochlea did not demonstrate any regeneration potential. However, a handful of studies using ototoxic drugs, such as aminoglycosides, have found evidence for limited proliferation within the adult utricular epithelia. The presence of these immature hair cells represents possible regeneration within the inner ear of mammalian animals. These potentially regenerated cells are not sufficient in quantity to restore function, but they indicate that cells within the inner ear have the potential to be regenerated if prompted through therapeutic means.

Two previous studies in hearing-loss animal models demonstrated that PBMT could increase the number of auditory hair cells within the cochlea after ototoxic gentamicin treatment. In the first study, organotypic cultures of cochlea from rats were given PBMT with an 810 nm laser diode at 8 mW/cm2 for 60 minutes a day for six days. Significant regrowth of cochlea hair cells was observed in laser-treated groups. Evidence of neural cell proliferation initiated by this laser therapy lends support to the regeneration of cochlear hair cells through PBMT since these are also formed from the neuroectoderm. Interestingly, there was no difference in the number of cochlear hair cells that received PBMT treatment without undergoing pre-exposure to a ototoxic drug, indicating that the regeneration induced by PBMT therapy may occur only after significant damage occurs to the hair cells within the cochlea. However, this study was done on cochlea that are still premature and may still have regeneration potential at this stage, whereas human cochlea hair cells are fully differentiated at birth.

A subsequent study from the same group used live adult rats that have mature cochlea subjected to ototoxic gentamicin treatment to study the effect of PBMT on cochlear hair regeneration. Rats irradiated with a laser power of 200 mW at 830 nm for 60 days for 10 minutes had a significant increase in the number of hair cells with a concomitant increased hearing threshold. Notably, hair cell growth did not reach normal numbers and was absent either where ototoxic damage was too severe, such as in the basal turn of the cochlea, or where ototoxic damage was too little, in the apical turn. These in vivo studies suggest that auditory hair cell growth by PBMT is possible for a certain section of hair cells in the cochlea, and only for a specific window of ototoxic damage.

Additionally, PBMT at 630 nm using LED was shown to enhance the differentiation of embryonic stem cells into inner ear hair-like cells. The mechanism attributed to this effect was PBMT-mediated downregulation of genes associated with neural development and the Hes5 gene, which normally inhibits the conversion of presensory cells into hair cells. Additionally, human utricular sensory epithelial cells (HUCs) were shown to undergo an epithelial to mesenchymal transition and could display features of a stem or progenitor-like state. This study indicates that sensory epithelia of the inner ear could potentially de-differentiate to have increased regenerative potential for generation of hair-cell progenitors. These in vitro studies support the regenerative capacity of cells within the inner ear, which could potentially be enhanced through application of PBMT. Further supporting this are in vitro studies demonstrating that mesenchymal stem cells (MSCs) of bone marrow origin from mice can differentiate into IHCs and OHCs when given specific growth factors in culture and forced expression of the transcription factor, Math1.

Species-specific requirements for the development of sensory progenitor cells from MSCs have also been observed. Human MSCs appear to require epidermal growth factor (EGF) and retinoic acid in culture for their directed differentiated into inner ear sensory cells. MSCs obtained from adipose tissue have also been shown to develop into hair cells through specific differentiation protocols. This approach bypassed the initial step of converting MSCs into otic progenitor cells prior to their differentiation into hair cells, thereby simplifying and speeding up the process of hair cell regeneration. Importantly, previous studies that provide evidence of hair cell regeneration from MSCs are based on in vitro approaches. Accordingly, in embodiments, the present invention provides a method for treating one or more of tinnitus, ear ringing, and sensorineural hearing loss, comprising intratympanic membrane injection of one or more specialized stem cells of mesenchymal origin into the inner ear, and modulating the injected stem cells through PBMT to direct differentiation into IHCs and OHCs.

PBMT in modulating cellular inflammation—SNHL can be caused by inflammation that occurs due to autoimmune diseases of the inner ear, or viral or bacterial infections that cause inflammation. In this context, anti-inflammatory agents have been employed in the treatment of sudden SNHL or autoimmune diseases of the inner ear. These treatment agents also include anti-TNF-α agents. Pro-inflammatory pathways, of which TNF-α is a major central player, are main mediators of cell death in auditory hair cells. Several pro-inflammatory proteins and signaling cascades that are responsible for promoting hair cell death are also modulated by PBMT. Accordingly, in embodiments of the present invention, reducing the activation of inflammatory pathways in auditory hair cells through PBMT protects hair cells from committing to cell death and promotes their survival instead. The ability of red light to modulate cytokines released from macrophages to reduce inflammation has been observed for many years. Additionally, joint inflammation was successfully treated in rat inflammation models treated with 50 mW or 100 mW PBMT using an 808 nm arsenide and aluminum gallium type diode. Treated rats had decreased pro-inflammatory molecules IL-6 and (IL)-113, with an even more pronounced effect with 50 mW compared to 100 mW treatment. However, 100 mW treated rats had a greater reduction of TNF-α compared to the 50 mW treatment. TNF-α reduction by PBMT has also been observed in wound healing animal models with high levels of inflammation.

Another example study examined the effect of PBMT on periodontal ligament cells that are implicated in periodontal disease, which is caused by chronic inflammation due to infection. PBMT using a 660 nm diode laser at 8 J/cm2 was found to exhibit a potent anti-inflammatory effect through reduction of lipopolysaccharide-stimulated expression of pro-inflammatory cytokines. This study showed that PBMT could decrease the expression of TNF-α, IL-6 and IL-8, and may work by downregulating the NF-κB signaling pathway. Collectively, the experimental evidence from these studies highlights the ability of PBMT to downregulate the same pro-inflammatory cytokines and proteins that are responsible for stimulating death of auditory hair cells in SNHL. PBMT appears to have the ability to switch cell fate from pro-death to pro-survival through modulation of these pathways.

The Food and Drug Administration (FDA) has cleared devices that use laser light in the red and NIR wavelength range, administered through a portable device, for the temporary relief of joint and muscle pain that causes chronic low back, neck, and shoulder pain. Clinical trials using these devices were more successful than using opioids or non-inflammatory steroidal anti-inflammatory (NSAID) medications to manage chronic musculoskeletal pain. This supports the ability of PBMT to reduce inflammatory responses within the body. Additionally, more than 200 devices that contain infrared light source or lamps to deliver topical heating have been cleared by the FDA under their Premarket Notification 510(k) process.

PBMT in wound healing—Early clinical evidence has demonstrated the benefits of PBMT in enhancing and/or accelerating wound healing in damaged tissues. The general mechanism appears to be through light-mediated infiltration of immune cells that are pro-inflammatory and promote the migration, adhesion and proliferation of fibroblasts. The expression of basic fibroblast growth factor (bFGF) is increased by PBMT. Wound sites are also found to close more quickly through the action of activated lymphocytes. In animal models, enhanced wound healing was demonstrated in a rat burn model using a superpulsed 904 nm laser through pro-inflammatory and anti-inflammatory effects. Treated rats had reduced inflammation, decreased expression of TNF-α, NF-κB, and upregulation of VEGF, FGFR-19541, HSP-60, HSP-90, HIF-1a, MMP-9 and MMP-2. Given the importance of wound healing in tympanic membrane (TM) perforations due to blast injury, these previous studies open an intriguing possibility of whether PBMT could benefit TM repair; TM perforations that rely on extensive wound healing for repair could also benefit from the stimulation of molecular factors that enhance wound healing by PBMT.

In clinical application, PBMT has emerged as a promising therapeutic approach for the treatment of oral mucositis that occurs in between 36 to 100% of cancer patients that undergo conventional treatment methods involving chemotherapy and/or radiation therapy. Oral mucositis is characterized by the development of oral sores that progress from erythema, ulceration, bleeding and necrosis according to stages outlined by the National Cancer Institute. The painful condition interferes with the ability of the patient to eat and can be life threatening in advanced stages if left untreated. The increasingly aggressive treatment methods for cancer with drugs like cisplatin and 5-fluorouracil have escalated the incidence of oral mucositis. Several clinical trials are currently in progress to study the efficacy of PBMT in the treatment and prophylactic prevention of oral mucositis (ClinicalTrials.gov identifier: NCT02682992). Meta-analysis of case studies and literature examining the effects of PBMT on oral mucositis have found that a dose of 2 J/cm2 for prevention and 4 J/cm2 for treatment in the red light wavelength range fulfills the criteria outlined by the Multinational Association of Supportive Care in Cancer (MASCC). Due to the numerous studies demonstrating the benefits of PBMT in treating oral mucositis, this approach is now an accepted therapy that is becoming more widely used. The MASCC and the International Society for Oral Oncology recently published guidelines recommending specific protocols for the use of PBMT in treating or preventing oral mucositis in cancer patients. Thus, PBMT is now recommended as one of the most effective approaches for clinical intervention of oral mucositis.

PBMT in the treatment of tinnitus and/or ear ringing—A handful of clinical studies have demonstrated the benefits of using PBMT in treating tinnitus. In one study using a 40 mW laser at 830 nm wavelength once a week for a total of ten weeks, up to 55% and 58% of patients with tinnitus found relief in the loudness and degree of annoyance of their symptoms, respectively. In another study, patients with tinnitus that were subjected to 5 mW soft laser at 650 nm for 20 minutes a day for 20 days had a reduction of symptoms in 49.1% of patients, and tinnitus disappeared in 18% of patients. In comparison to other treatment methods, PBMT appears to be a treatment approach that can be further developed for even more efficacy. More likely, repeated treatments would be necessary for treating tinnitus as indicated in a study which found that PBMT was effective in short-term treatment. Cellular mechanisms involved in PBMT repair of tinnitus remains unclear but appear to involve the established paradigm that light therapy stimulates ATP production and activates mitochondria within the hair cells that stimulate further repair processes within the inner ear.

Applications of PBMT to cochlear hair cells for the prevention or treatment of hearing loss and tinnitus and/or ear ringing. To date, there are no therapeutic treatment methods available to restore hearing loss in most cases. Hearing aids are not usually beneficial for SNHL since they rely on IHCs to respond to sound waves, and may cause further damage due to the amplification of sound. Instead, surgically implanted cochlear implants that directly stimulate the auditory nerve without relying on IHCs have had moderate success in partially restoring hearing loss due to SNHL. While cochlear implants can substantially improve the quality of life of individuals affected by SNHL, they do not work in cases where the spiral ganglion is damaged since they stimulate this component to improve hearing. As hearing loss that results from loss of auditory hair cells has been associated with other pathophysiological complications like the progressive degeneration of auditory neurons, it is necessary to find treatment methods that can tackle the source of SNHL to prevent further complications such as these from arising.

Recent advancements in restoring hearing loss have focused on regeneration of auditory hair cells through molecular approaches that include gene therapy, stem cell therapy, and gene editing techniques. Since mammalian auditory cells cannot regenerate themselves, research to develop new treatment methods for hearing loss has focused on regeneration of hair cells through endogenous stem cells or generation of hair cells from surrounding supporting cells within the cochlea. Additionally, some interest has been on inducing cellular proliferation within pre-existing mature hair cells and their surrounding cells. Of these, regeneration of hair cells through targeting their supporting cells is considered to be the most promising option in which supporting cells are reverted back to a progenitor-like state followed by selective differentiation into hair cells. One recent example is the generation of hair cells from supporting cells through injection of an inhibitor of gamma-secretase into the inner ear; this approach restored hearing loss in mice. However, molecular based approaches face significant hurdles in translational application including ambiguity of the functionality of regenerated hair cells, limitations in delivery methods of molecular approaches, and gaps in knowledge of the underlying mechanisms of auditory hair cell regeneration that are necessary to develop safe and effective therapeutic approaches. Therefore, alternative approaches to restore or prevent cochlear hair cell loss that could be easily translated to the clinic are necessary.

Cellular mechanisms of PBMT to treat SNHL—The use of PBMT for the prevention or treatment of hearing loss that is caused by damage to the auditory hair cells is proposed. PBMT is an established treatment method for promoting tissue protection, restoration and healing that is safe, non-invasive and potentially has no side effects. Its potent anti-inflammatory, pro-survival and pro-proliferative effects have been demonstrated in hundreds of studies to date, in both animal models and human clinical studies. Mechanistically, PBMT stimulates biochemical pathways within the cell to enhance cellular energy and promote its healing and regenerative effects.

In the application of PBMT for treatment of SNHL, PBMT has shown promising pre-clinical data in increasing cochlear hair cell growth in animal models with hearing loss induced by ototoxicity and it is already an approved therapy for the treatment of adult pattern hair loss. Preliminary studies have already shown that the application of PBMT reduces toxicity cause by gentamicin treatment in an in vitro model of auditory hair cells. This study also provided mechanistic data to support that the increased mitochondrial membrane potential and higher ATP levels within cells elicited by PBMT treatment was responsible for protection of hair cells from apoptosis after gentamicin treatment.

At the cellular level, the use of PBMT in the potential application of cochlear hair protection and/or regrowth is also supported through numerous published pre-clinical studies. PBMT downregulates pro-inflammatory and pro-apoptotic proteins in various cell types that are established in mediating death of auditory hair cells in the cochlea. Another potential mechanism of PBMT-mediated protection of auditory hair cells may be the link between PBMT—mediated increase in ATP levels and possible increased adenosine signaling through the A1 receptors that promotes survival of auditory hair cells. While this link has not yet been directly investigated, additional research on whether PBMT affects adenosine signaling in cochlear hair cells to protect them from cell death is needed to provide more information on cellular mechanisms of how PBMT could repair auditory hair cell function to benefit SNHL.

Another area that warrants further investigation is the seemingly contradicting role of ROS in auditory hair cell death and in PBMT. While generation of ROS is an established cellular contributor to mediating death of auditory hair cells, PBMT also exerts its therapeutic influence through elevation of ROS. Generally, ROS is considered to have a biphasic dose response in cells, with low levels of ROS exerting beneficial effects and high levels of ROS being toxic. Several lines of evidence could provide an explanation for how PBMT could be beneficial for auditory hair cell survival. First, ROS production by PBMT is highly dependent on the wavelength of light that is used. Accordingly, previous studies have found that PBMT up to 5 J/cm2 could increase proliferation and wound healing of fibroblasts, but fluences above 16 J/cm2 at the same wavelength caused excessive oxidative stress. Conversely, PBMT at 825 nm with a fluence of 5 J/cm2 created as much ROS levels as a fluence of 15 J/cm2, 20 J/cm2 and 25 J/cm2, demonstrating the effect of wavelength on ROS production. Along these lines, a multi-wavelength protocol could be beneficial in clinical applications of PBMT to provide the cellular benefits of PBMT without generation of high levels of ROS. Furthermore, multiple research studies have demonstrated that low energy densities produce minimal levels of ROS that induce cell proliferation, differentiation, and anti-apoptotic event, while high energy densities produce high levels of ROS that are pro-apoptotic.

PBMT mediated modulation of cell signaling pathways to treat SNHL—Nevertheless, the overwhelming benefits of PBMT in hair cell growth are established. Applying PBMT to auditory hair cell protection and growth would work by the same general principles as stimulation of the hair follicle in the treatment of adult pattern hair loss by PBMT, but with focus on stimulation of auditory hair cell growth.

Another potential link between stimulating the regeneration of auditory hair cells is through its supporting cells. the control of sensory progenitor cells in the inner ear through specific signaling pathways within the mammalian cochlea. Mitotic reentry into the cell cycle is an important step in the ability of the hair cell to regenerate. Normally, after the completion of development, the progenitor cells within the Organ of Corti in the mammalian cochlea lose their ability to proliferate through exiting the cell cycle, an event that is highly dependent on the function of the gene, Cdkn 1b. Re-entry into the cell cycle is an important component of cellular regeneration that facilitates proliferation of auditory hair cells in non- mammalian animals in which restoration of hearing after hearing loss occurs. While the Wnt signaling pathway is implicated in driving proliferative responses within the cochlea including in the post-natal mammalian ear, the Hippo signaling pathway represses growth and proliferation of cells to oppose the function of the Wnt pathway.

An important component of the Hippo pathway is its downstream effector proteins YAP/Tead, which have been implicated in stimulating proliferation of hair cells during development. PBMT has been linked to modulation of YAP through a study demonstrating that PBMT at 2 J/cm2 fluence prevented amyloid-β-peptide mediated apoptosis in an in vitro model of Alzheimer's disease through preventing the translocation of YAP into the nucleus. This study provides evidence for the ability of PBMT to influence the Hippo/YAP pathway, which could potentially be applied to re-establishing proliferative potential and protection against apoptosis in the inner ear.

Cell signaling through the Wnt pathway occurs in concert with the actions of YAP to promote proliferation within the inner ear. The Wnt signaling pathway is one of the most important molecular determinants of formation of inner ear sensory epithelia during early development. The expression of the transcription factor, Atohl, which is necessary and sufficient for the differentiation of hair cells in the inner ear, is regulated by the Wnt signaling pathway. Inhibition of the Wnt pathway blocks the proliferative capacity of prosensory cells and its reactivation can promote proliferation again. The Wnt/β-catenin pathway is also crucial for the growth and morphogenesis of the hair follicle in hair regrowth. PBMT at 655 nm wavelength can facilitate the growth of human hair through activation of the Wnt signaling pathway, further supporting PBMT as a therapeutic approach to promoting hair cell growth.

The Fibroblast Growth Factor (FGF) signaling pathway is another major component of auditory hair cell and supporting cell differentiation during cochlear development. Basic Fibroblast growth factor (bFGF) is also known to play a role in the protection of auditory hair cells from acoustic trauma and functions in the regeneration of cochlear hair cells after damage in non-mammalian animals. Interestingly, bFGF changes its cellular distribution within hair cells after noise exposure. Numerous studies have demonstrated that bFGF is one of the major growth factors to be released after PBMT. The total amount of bFGF released displayed a dose response that increased the amount of bFGF released when exposure times and number of treatments were increased. Therefore, stimulation of bFGF is another mechanism through which auditory hair cells could be protected after ototoxicity and/or acoustic trauma through application of PBMT.

Novel ways to treat tinnitus and/or ear ringing and other middle and inner ear disorders through PBMT—Other middle and inner ear disorders could also potentially benefit from the use of PBMT as a therapeutic approach. Based on the few studies available, PBMT could be further optimized for the treatment of tinnitus and/or ear ringing as some studies have shown promising results.

Applications of PBMT in the potential treatment of tinnitus in human patients have shown mixed results. Some studies have found positive results in dissipating symptoms of tinnitus through PBMT treatment. Apart from these few studies, the effect of PBMT on hearing loss and other middle and inner ear disorders has not been investigated. As the success of PBMT is highly dependent on multiple dosing variables such as energy density, irradiation, pulsed light wave, continuous wave, exposure time, and area of treated skin tissue and distance from light to skin tissue, the thorough optimization of these factors is necessary for this therapeutic approach to be beneficial.

Recently, a handful of studies have provided evidence suggesting that PBMT can be used as a form of non-invasive brain stimulation, which is termed transcranial brain stimulation (TBM). This technique delivers light energy into the brain through the use of PBMT, which can provide multiple benefits such as increased ATP production, blood flow and availability of oxygen within the brain. Additionally, the ability of PBMT to repair damaged neurons has also been documented, providing the basis for several ongoing clinical trials testing the effect of PBMT on recovery after brain injury and as a therapy for other brain disorders. A pilot clinical study examining the effect of pulsed light at 40 Hz, 810 nm wavelength and 240 J/cm2 energy density per treatment session found that neural oscillations were significantly increased in 20 treated individuals without the occurrence of any adverse effects, supporting the investigation of PBMT for the treatment of clinical conditions in which modulating neural plasticity could be beneficial. As tinnitus is a condition that is caused by maladaptive neural plasticity with visible auditory neural changes in patients with tinnitus, such as a change in the peak time of firing between neurons in the dorsal cochlear nucleus, the use of PBMT could potentially mitigate symptoms associated with the condition. Stimulation of the dorsal cochlear nucleus through combined auditory and somatosensory means using small bursts of sound followed by electrical stimulation delivered transcranially alleviated symptoms of tinnitus in research with guinea pigs and humans. Therefore, the combined use of PBMT with electrical stimulation, acoustical stimulation, or combined in various forms, is proposed herein. These treatments could also benefit patients suffering from tinnitus and studies have supported the development of bi- and/or tri-modal optical stimulation of the cochlea with NIR light with electrical stimulation and acoustical stimulation. The role of electrical stimulation in mitigating chronic pain has had a limited investigation, however some recent clinical trials researching their combined benefit have been recently registered (ClinicalTrials.gov identifier: NCT04020861).

Otosclerosis is a condition in which bones of the middle ear are abnormal, causing functional disturbances to structures within the ear. Approximately one-third of all people with otosclerosis develop SNHL that occurs before the onset of age-related hearing loss. One of the main features of otosclerosis is the loss of auditory hair cells. Both IHCs and OHCs are lost in this condition. Recent studies have found that the hardness of the cochlear bone matrix causes hearing loss. Mechanistically, dysregulation of the TGF-β signaling pathway, an important component of osteoblast differentiation and integrity, disrupts the normally hard cochlear bone and leads to hearing loss. Several lines of evidence have supported the ability of PBMT to promote bone healing and restore hardness to bones in areas where low-level light is applied and TGF-β is one of the proteins known to be modulated by PBMT. These preliminary studies indicate another novel angle from which PBMT may benefit hearing loss in cases of otosclerosis and/or compromised cochlear bone integrity.

Potential limitations of PBMT in clinical therapeutic application are that repeated treatments are usually required. In specific application of PBMT for treating and/or preventing hearing loss, this could cause inconvenience for patients, driving non-compliance especially for those with extensive hair cell loss and/or severe damage to hair cells that will likely require multiple treatments. However, PBMT is considered to have little no side effects, so numerous treatment sessions do not have any other disadvantages other than the time commitment required and potential cost to the patient.

As SNHL may be caused by a variety of factors, a therapeutic strategy that involves a combination of other adjunctive therapeutic drugs, supplement, biologic compounds, and/or cells (e.g., antioxidant drugs and a mode of PBMT that is optimized to direct the regeneration of MSCs and/or supporting cells into hair cells) with PBMT is proposed herein. A multi-wavelength protocol that activates necessary signaling pathways and molecules can facilitate proliferation and differentiation of MSCs and/or progenitor cells that are directly injected into the ear to become hair cells, through influencing the steps that commit them to these pathways.

In summary, the cellular mechanisms elicited by PBMT could prevent auditory hair cell death from being stimulated when they undergo a stress trigger. Instead, auditory hair cells will commit to cellular pathways of proliferation, survival and growth. Based on the abundance of evidence, PBMT has shown the capability to prevent further and possibly restore SNHL hearing loss through treatment and/or prevention of cochlear hair cell loss. With this therapeutic method, it will be possible to treat the cause of SNHL rather than just symptoms.

Previous failures by other groups constitute evidence of a long-felt need for effective treatment and prevention of hearing loss, particularly SNHL and tinnitus and/or ear ringing. While some in vitro studies have evaluated hair cell regeneration using mesenchymal stern cells (MSCs), the in vivo translation of such approaches requires further research and validation, due in part to the sensitivity of MSCs to their microenvironment and the relative unpredictability of MSC differentiation in vivo. In addition, while previous efforts have been made to utilize PBMT for treatment of tinnitus, no statistically significant difference between treated and untreated groups was observed. As such, there has been some degree of unpredictability about the feasibility of utilizing PBMT for treatment and/or prevention of hearing loss, particularly SNHL, tinnitus and/or ear ringing.

Other conditions are characterized by a long-felt need for effective treatment and prevention. Oral mucositis, a type of mucositis which occurs in the mouth and is painful and debilitating, is a side effect for cancer patients undergoing chemotherapy and/or radiation therapy. There are limited treatment options for oral mucositis, and because treatment often consists of managing the mucositis after diagnosis rather than prophylactically treating the tissue to prevent and/or reduce the severity of the mucositis, it is evident that there is a need for improved therapies and interventions to treat mucositis, particularly oral mucositis.

Accordingly, there is a need for improved and non-invasive localized treatments for a variety of conditions, including but not limited to SNHL, tinnitus, ear ringing, and oral mucositis, which may be combined with other localized and/or systemic treatments where appropriate. The present invention addresses this unmet need.

In this respect, before explaining at least one embodiment of the System and Method for Photobiomodulation in greater detail, it is to be understood that the design is not limited in its application to the details of construction and to the arrangement of the components set forth in the following description and illustrated in the drawings. The System and Method for Photobiomodulation is capable of other embodiments and of being practiced and carried out in various ways. Also, it is to be understood that the phraseology and terminology employed herein are for the purpose of description and should not be regarded as limiting.

### SUMMARY OF THE INVENTION

The primary advantage of the Systems and Methods for Photobiomodulation is the ability to apply the optimal quality and quantity of therapeutic light to the portion of the subject to achieve specific therapeutic outcomes. For example, the photon source device may be fitted to the subject, such that a distance between a light source of the photon source device and the portion of the subject to be irradiated is definite, which allows the intensity and coverage of the therapeutic light to be adjusted, known, and consistently applied for each treatment. In addition, because a biological process may be stimulated, accelerated and/or inhibited by PBMT, control of the wavelength, power (irradiance), time of exposure, illumination sequence, area illuminated, and depth of penetration of the therapeutic light delivered to the portion of the subject may be adjusted to produce different treatment modes, one or more of which may be appropriate for a particular state of the condition, disease, and/or disorder.

Another advantage of the Systems and Methods for Photobiomodulation is the ability to diagnose, evaluate, or diagnose and evaluate a condition, disease, and/or disorder treatable by PBMT and/or other related physiological bio-parameters. The invention provides methods for evaluating one or more physiological parameters, or states, of the subject, both before, during and/or after PBMT treatment, to determine the effectiveness of the PBMT treatment and/or status of the subject. In embodiments, such methods may be performed by a person such as a healthcare worker, the control system of the invention, and/or both the person and the control system. In embodiments, such methods may be performed by the subject receiving the treatment, with little or no assistance from the healthcare worker.

Yet another advantage of the Systems and Methods for Photobiomodulation is that it provides a PBMT system comprising a photon source device which comprises one or more light sources configured to deliver an optimal quantity of a therapeutic light based upon the state of a subject's disease and/or disorder to a portion of the subject, and a control system operably connected to the photon source device, such that the control system is configured to control the photon source device. The invention may be utilized to mitigate further loss of sensorineural hearing, including mitigating loss of detection of auditory sound frequency and intensity ranges. The invention may he utilized to restore previously impaired and/or lost sensorineural auditory sound frequency and intensity ranges. In addition, the invention may be used to stimulate and/or inhibit underlying physiology to prevent further loss of auditory acuity, to restore lost sensorineural auditory frequency and/or intensity ranges, or any combination thereof.

A further advantage of the Systems and Methods for Photobiomodulation is that the control system may comprise a non-transitory computer-readable storage medium with instructions encoded thereon which, when executed by a processor, causes the PBMT system to perform a method. In embodiments, the method may comprise receiving a first evaluation of a physiological state of the subject and compiling a first signature from data of the first evaluation, delivering the therapeutic light to the portion of the subject, receiving a second evaluation of the physiological state of the subject and compiling a second signature from data of the second evaluation, and comparing the first signature with the second signature to determine the probability of a change in the physiological state.

Another advantage of the Systems and Methods for Photobiomodulation is that the method of the control system may further comprise adjusting the output of the therapeutic light (e.g., wavelength, irradiance, time of exposure, illumination sequence or a combination of all), adjusting the area illuminated of the subject which receives the therapeutic light, or a combination of all. In embodiments of the PBMT system, the photon source device may be fitted to the subject, such that a distance between the light source and a selected location on the subject is controlled to define the optimal quantity of the therapeutic light delivered. Accordingly, in embodiments of the PBMT system, the photon source device may have a feature that custom fits into an ear of the subject to optimally deliver the therapeutic light to and through a tympanic membrane into the cochlea of the subject, and/or alternatively, may have a feature that custom fits into a mouth of the subject to optimally deliver the therapeutic light to the selected mucosal membrane locations in the mouth of the subject.

Yet another advantage of the Systems and Methods for Photobiomodulation is that a portion of the subject may comprise an exogenous material. In embodiments, the exogenous material may comprise a stem cell, an adjunctive therapeutic compound and the PBMT system of the present invention may be used to overcome certain limiting factors to improve the effectiveness of the stem cell and/or adjunctive therapy. In this manner, the PBMT may be additive to the stem cell/adjunctive therapy, or the stem cellladjunctive therapy may be additive to the PBMT, according to needs in a particular scenario.

A further advantage of the Systems and Methods for Photobiomodulation is that the invention provides a photon source device for photobiomodulation, comprising one or more light sources configured to deliver an optimal quantity of a therapeutic light therefrom, a plurality of sensors configured to detect that the photon source device is optimally located for use, and a control system that operably connects a power source to the light source. In embodiments of the photon source device, the plurality of sensors may comprise a placement sensor which detects if the photon source device is placed in a proper location to optimally deliver the requisite optimal therapeutic light to the body area, e.g., a tympanic membrane, cochlea and/or an oral mucosal tissue, of a subject. In embodiments of the photon source device, the plurality of sensors may comprise a proximity sensor which measures a distance between the light source and a selected portion of a subject (e.g., distance sensor such as acoustic and/or optical time of flight sensor), or an imaging array that detects the pathway the light pathway the light source would illuminate.

Another advantage of the Systems and Methods for Photobiomodulation is that the photon source device may be wearable by a subject, such that if the photon source device is worn by the subject, the plurality of sensors may detect that the photon source device is positioned for use and/or optimal delivery of therapeutic light.

Yet another advantage of the Systems and Methods for Photobiomodulation is that the photon source device may comprise one or more light modulators. The light modulators may convert a first wavelength of light into one or more second wavelengths of light to produce the optimal therapeutic light. Exemplary light modulators which may be utilized for this purpose include one or more waveguides, one or more filters, one or more quantum dots, or any combination thereof. In embodiments of the photon source device, the photon source device may comprise a control system operably connected to the photon source device, and the control system may be at least partially integral with the photon control device and be configured to control at least part of the photon source device.

A further advantage of the Systems and Methods for Photobiomodulation is that the invention provides a method for photobiomodulation, comprising: evaluating a physiological bioparameter, and/or state, of a subject and compiling a first signature from data of the first evaluation, positioning a photon source device within and/or adjacent to the subject, activating the photon source device to deliver an optimal quantity of a therapeutic light to a portion of the subject, evaluating the physiological bioparameter, and/or state of the subject and compiling a second signature from data of the second evaluation, and comparing the first signature with the second signature to determine a change and/or the probability of change in the physiological state. In embodiments of the method, the method may further comprise adjusting the quantity of the therapeutic light, adjusting the area illuminated on the subject which receives the therapeutic light, or both. The system may also utilize external data to develop the first and/or subsequent state of the subject.

Another advantage of the Systems and Methods for Photobiomodulation is that the method may further comprise administering an exogenous material to the subject. Exemplary exogenous materials include treatments such as localized and/or systemic therapies, including but not limited to supplements, pharmaceutical compositions, biological compositions, cell-based therapies, and any combination thereof. In embodiments, the exogenous material may comprise a stem cell. In embodiments of the method, the photon source device used in the method may comprise a light source configured to deliver the optimal quantity of the therapeutic light therefrom, a plurality of sensors configured to detect that the photon source device is optimally located for use, and a control system that operably connects a power source to the light source. In embodiments of the method, the photon source device used in the method may be fitted to the subject, such that a distance between the light source and the portion of the subject is controlled to define the quantity of the therapeutic light delivered.

Another advantage of the Systems and Methods for Photobiomodulation is to provide systems, devices, and methods that enable the effective treatment of a variety of conditions, diseases, and disorders using photobiomodulation therapy. Another object of the invention is to provide photon source devices which may be effectively controlled to provide localized PBMT, optionally in combination with one or more other localized therapies, one or more other systemic therapies, or both.

Another advantage of the Systems and Methods for Photobiomodulation is that it provides for non-invasive therapy, regional therapy, periodic therapy , continuous therapy, episodic therapy to prevent and/or restore acute hair and supporting cell impairment, therapy combined with other therapeutic devices or exogenous compounds including supplements, drugs, biologics and genetics, non-invasive therapy, regional therapy, periodic therapy e.g. not daily, continuous, episodic therapy to prevent and/or restore acute hair and supporting cell impairment. The therapy can be combined with other therapeutic devices and/or exogenous compounds (supplements, drugs, biologics, genetic), the therapy can be automated so no third party intervention is required to enable optimal therapy, and/or automated diagnostic sensing of bioparameter can drive automated therapeutic adjustment and efficacy, and/or automated diagnostic sensing to measure therapy efficacy, and/or automated diagnostic subject compliance measurements to therapy schedule.

Another advantage of the Systems and Methods for Photobiomodulation is that it provides for a data management and analytics system which use diagnostic data from device and/or external devices and sources to adjust schedules for the subject's therapy and diagnostics. The diagnostic data can be generated from the device before, during or after current and/or past measurement cycles, as well as other diagnostic devices used before, during or after current and/or past device use schedules. Other analytic data the system can utilize include data from the subject or other subjects past medical history, environmental exposure, diagnostic sensors, therapies previously received, other medical procedures, other medical devices. The data management and analytic system features can be integrated into the device, into an adjacent device e.g. cell phone, smartphone, tablet, computer. The data management and analytic system, diagnostic and therapy functions can be integrated into one or more devices. The system's diagnostic and therapy functions can be separate or combined and/or integrated into other devices, e.g. handheld device, ear pods, hearing-aids, head phones, personal sound amplification devices, sound protection devices.

Another advantage of the Systems and Methods for Photobiomodulation is that it provides a system architecture that integrates data from the human/biointerface devices performing therapies and diagnostics with external data sets, manual data inputs within APPS and data management and analytic system. The system architecture may employ analytics in a manual and automated fashion using algorithms, artificial intelligence and machine learning at one or more locations within the system

Another advantage of the Systems and Methods for Photobiomodulation is that it provides a setup/authorization process which allows a subject and/or third party to create a subject profile within an application (APP), automatically import external data, pair a device with a subject profile, and pair an adjacent device, e.g. cell phone, computer, tablet etc., using the APP located on a phone, tablet and/or computer. The system utilizes the data from the setup process to creates diagnostic and therapeutic schedules for the subject, authorizes the device to be used, creates and periodically updates a custom therapy protocol for the subject, enables the therapy protocols to be automatically installed into the device from the APP and/or data management system.

Another advantage of the Systems and Methods for Photobiomodulation is that it provides for one or more hearing protection and/or restoration configurations including diagnostic and therapy schedules and customized therapy protocols with only device placement diagnostic sensing enabled.

Another advantage of the Systems and Methods for Photobiomodulation is that it provides for one or more hearing protection and/or restoration configurations including diagnostic and therapy schedules and customized therapy protocols with all diagnostic sensing integrated into the device and APP enabled.

These together with other advantages of the Systems and Methods for Photobiomodulation, along with the various features of novelty, which characterize the design are pointed out with particularity in the claims annexed to and forming a part of this disclosure. For a better understanding of the Systems and Methods for Photobio-modulation its operating advantages and the specific objects attained by its uses, reference should be made to the accompanying drawings and descriptive matter in which there are illustrated the preferred and alternate embodiments of the Systems and Methods for Photobiomodulation. There has thus been outlined, rather broadly, the more important features of the design in order that the detailed description thereof that follows may be better understood, and in order that the present contribution to the art may be better appreciated. There are additional features of the Systems and Methods for Photobio-modulation that will be described hereinafter, and which will form the subject matter of the claims appended hereto.

The preferred embodiment of the Systems and Methods for Photobio-modulation will provide a photobiomodulation therapy (PBMT) system which comprises one or more photon source devices and a control system for controlling the photon source device, including an on body device to deliver therapy, an on body device to deliver therapy and perform diagnostics, an on body device to perform diagnostics, an adjacent device to perform system features and functions not on the on body device, and a remote data management and analytics system to perform system features and functions not on adjacent device and/or on body device. The PBMT system may be configured to deliver a quantity of a therapeutic light from ˜280 to ˜1000 nanometer (nm) wavelength interval, such as light that comprises red light (620 to 750 nm), near-infrared (near-IR) light (750 nm to 3 μm), and/or combinations of various wavelengths of light in selected regions of the electromagnetic spectrum to a portion of a subject, such as a tissue surface, membrane, or mucosal membrane. In embodiments, the PBMT systems, devices, and methods may utilize one or more light wavelengths including, but not necessarily limited to: 447 nm, 532 nm, 635 nm, 808 nm, and any combination thereof. The therapeutic light benefits the subject by stimulating and/or inhibiting one or more physiological responses of the area illuminated by the light, e.g., by accelerating or slowing one or more regional or systemic biological processes, or by both accelerating and slowing one or more regional or systemic biological processes over time.

In alternate embodiments of the Systems and Methods for Photobiomodulation primary elements will include as prominent configurations, design and operational functions:

Element 1—one or more light sources which are therapeutic energy adjusted for location on the subject for optimal therapy results.

Element 2—one or more light sources which are therapeutic energy adjusted from previously performed diagnostic test results data for optimal therapy results.

Element 3—one or more light sources in which therapeutic energy is adjusted when device location changes on the body during therapy.

Element 4—elements 1-3 above in varying combinations.

Element 5—elements 1-4 above light sources wavelengths are adjusted for optimal therapy results.

Element 6—elements 1-4 vabove wherein the light sources energy output is adjusted for optimal therapy results.

Element 7—elements 1-4 above wherein the area of body illuminated by light energy is adjusted for optimal therapy results.

Element 8—elements 5-7 above in varying combinations.

Element 9—elements 1-8 above with one or more of following diagnostic capabilities:

(a) Auditory Tests: evoke potential tests. e.g. auditory brainstem response (ABR) and/or auditory steady-state response—ASSR, otoacoustic emissions (OAE), Pure-Tone, Speech Testing, Word tests e.g. Words in Noise, Digits in Noise, tests of the middle ear;

(b) Physiological: Temperature (e.g. ear, skin, tissue, core), tissue bioimpedance, electroencephalogram—EEG, heart rate, heart rate variability, SpO2, StO2, blood pressure, pulse wave velocity, respiration rate, tissue composition, motion, ambient noise, otitis media, cerumen, optical ear canal and tympanic membrane topography scans and/or 2D and/or 3D images and/or models, or other electrical, optical or mechanical physiological measurements.

Element 10—elements 1-9 above with an advanced analytics capabilities system and/or device generated diagnostics and/or therapy data.

Element 11—elements 1-10 above with an advanced analytics capabilities system and/or device generated diagnostic and/or therapy data, and/or externally input data, and/or imported external data.

Element 12—elements 1-11 above analytic data output that adjusts diagnostic and therapeutic schedules based on prior analyzed data sets from the subject and/or other subjects.

Element 13—elements 1-12 above analytic data output that adjusts therapeutic PBMT protocols based on prior analyzed data sets from the subject and/or other subjects.

Element 14—elements 1-13 above data management system generated data for review by subject and/or authorized third party.

Element 15—elements 1-14 above combined with one or more other therapies such as:

(a) Exogenous chemicals e.g. pharmaceutical drugs, biologics, gene therapies e.g. stem cells, supplements;

(b) Devices—hearing aids, sound amplification; noise protection, communication devices, therapeutic devices;

(c) Services—Acupuncture, surgery, meditation, auditory training, brain plasticity remodeling training.

Element 16—elements 1-15 above fully integrated into one or more devices on the body—ear pod, headphone, noise protection, hearing-aid, personal sound amplification, communication devices.

Element 17—elements 1-15 above with system features and functions located on an on-body device and one or more adjacent computing devices, e.g. smartphone, computer, tablet or similar.

Element 18—elements 1-15 above with system features and functions located on an on-body device, and one or more adjacent computing devices. and one or more remote data management and analytic systems

Element 19—elements 1-18 above with one or more data management and analytic systems that manually and/or automatically escalate subject care interventions utilizing data from current and/or prior diagnostic and therapy data analysis by one or more of the system analytic features. These interventions can be one or more of the following: Send one or more electronic/digital communication notifications (text, email, voicemail, etc.) to one or more authorized third parties for review and/or action; Automatically create a notification to review analyzed and historical data within data management system by one or more authorized third parties; Automatically scheduling an appointment and/or meeting with subject and authorized third party either in person or through other electronic/digital means, e.g. telemedicine, virtual presence, telephonic or televideo.

Element 20—elements 1-19 above with automated methods and features to enable manual or automated payment invoicing to authorized third parties for services provided, subscriptions and/or other goods and services, e.g. insurance, health savings accounts, credit/debit cards, employers, government agencies, individual service providers, etc.

Element 21—elements 1-19 above with automated and manual methods and procedures to transfer data created, analyzed, imported and/or stored within a data management system to subject and/or authorized third parties.

With respect to the above description then, it is to be realized that the optimum dimensional relationships for the parts of the Systems and Methods for Photobiomodulation, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present design. Therefore, the foregoing is considered as illustrative only of the principles of the Systems and Methods for Photobiomodulation. Further, since numerous modifications and changes will readily occur to those skilled in the art, it is not desired to limit the Systems and Methods for Photobiomodulation to the exact construction and operation shown and described, and accordingly, all suitable modifications and equivalents may be resorted to falling within the scope of this application.

### BRIEF DESCRIPTION OF THE DRAWINGS

The accompanying drawings, which are incorporated in and form a part of this specification, illustrate embodiments of the Systems and Methods for Photobiomodulation and together with the description, serve to explain the principles of this application.

FIG. 1 depicts a cross-sectional schematic view of the photobiomodulation device inserted in a subject's ear canal.

FIG. 2 depicts a photobiomodulation device configured to apply the device diagnostic and therapeutic capabilities to one or both of a subject's ears, including a light source in communication with a smartphone or like device, both of which are connected to the two photobiomodulation devices.

FIG. 3 depicts a detailed cross-sectional view of a subject's ear anatomy with a photobiomodulation device inserted into the ear canal.

FIG. 4 depicts an enlarged cross-sectional detailed view of a photobiomodulation device which operates and communicates wirelessly and is powered by an on-board battery.

FIG. 5 depicts an enlarged cross-sectional detailed view of a photobiomodulation device which operates and communicates wirelessly and is powered by an on-board battery as well as an external wired power source.

FIG. 6 depicts an enlarged cross-sectional detailed view of a photobiomodulation device which operates and communicates wirelessly, has an external light source connection, and is powered by an on-board battery as well as an external wired power source.

FIG. 7A depicts a photobiomodulation device configured in a dual device for insertion into one or both of a subject's ears, including a light source in communication with a smartphone or like device, both of which are connected to the two photobiomodulation devices.

FIG. 7B depicts a photobiomodulation device configured in a head set style dual device for insertion into both of a subject's ears, including a light source in wireless communication with a smartphone or like device.

FIG. 7C depicts a photobiomodulation device configured in a head set style dual device for insertion into both of a subject's ears including a light source in communication with a smartphone or like device, both of which are connected to the two photobiomodulation devices.

FIG. 8 depicts a schematic diagram of the photobiomodulation system bus illustrating the numerous communications capabilities between the system bus and the hardware elements integrated into the photobiomodulation device.

FIG. 9 depicts a schematic diagram of the various telecommunications capabilities of the PBMT device either alone or coupled to a smartphone utilizing a smartphone application (APP) or other like computing device.

FIG. 10 depicts a flow chart illustrating the system architecture interrelationships between the human/biointerface, the photobiomodulation device and the external data sets and inputs which are cloud based and located on a smartphone application (APP), for facilitating analytics performed by the photobiomodulation system.

FIG. 11 depicts a flow chart illustrating the setup/authorization steps in which a subject or authorized third party can create a subject profile, import external data and pair an external device to create diagnostic and therapeutic protocols.

FIG. 12 depicts a flow chart illustrating the steps taken in a hearing restoration and/or protection configuration having the diagnostic and therapeutic functionality initiated with only insertion location sensing capability enabled in the photomodulation device.

FIG. 13 depicts a flow chart illustrating the steps taken in a hearing restoration and/or protection configuration having the diagnostic and therapeutic functionality initiated with all sensing capabilities enabled in the photomodulation device.

### DETAILED DESCRIPTION OF THE PREFERRED EMBODIMENTS

As required, the detailed embodiments of the present Systems and Methods for Photobiomodulation 10A, 10B, 10C, 10D, 100, 200 and 300 are disclosed herein, however, it is to be understood that the disclosed embodiments are merely exemplary of the design that may be embodied in various forms. Therefore, specific functional and structural details disclosed herein are not to be interpreted as limiting, but merely as basic for the claims and as a representative basis for teaching one skilled in the art to variously employ the present design in virtually any appropriately detailed structure as well as combination.

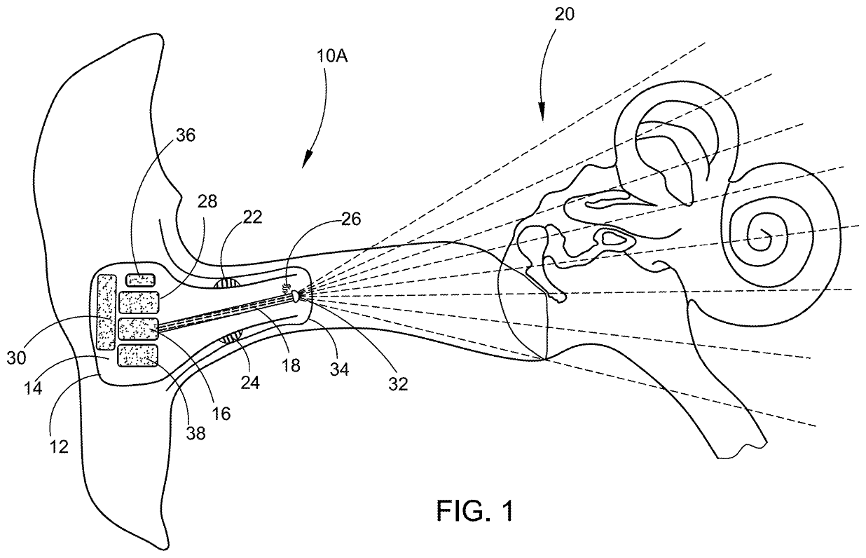

Referring now to FIG. 1, there is depicted a cross-sectional schematic view of an exemplary photobiomodulation system photon source device 10A inserted into an ear canal of a subject. Generally, a photobiomodulation system photon source device 10A comprises a housing 12 with an interior section 14 including a light source 16 and a waveguide 18 configured to emit a quantity of a therapeutic light 20 in one or more wavelengths therefrom. A plurality of placement sensors 22 and 24 configured to detect that the photobiomodulation system photon source device 10A is properly positioned for use, and a proximity sensor 26 are connected to a control system 28 that operably connects a power source 30 to the light source 16. The photobiomodulation system photon source device 10A may also include a light modulator 32 on the end of waveguide 18 which illuminates light outward from aperture 16 in the device protective cover 34. The photobiomodulation system photon source device 10A may also include a microphone/receiver 36 and speaker 38, and comprise one or more means for aiming and/or guiding the therapeutic light down the ear canal for delivery to the middle ear and/or the inner ear (see details of this function discussed below).

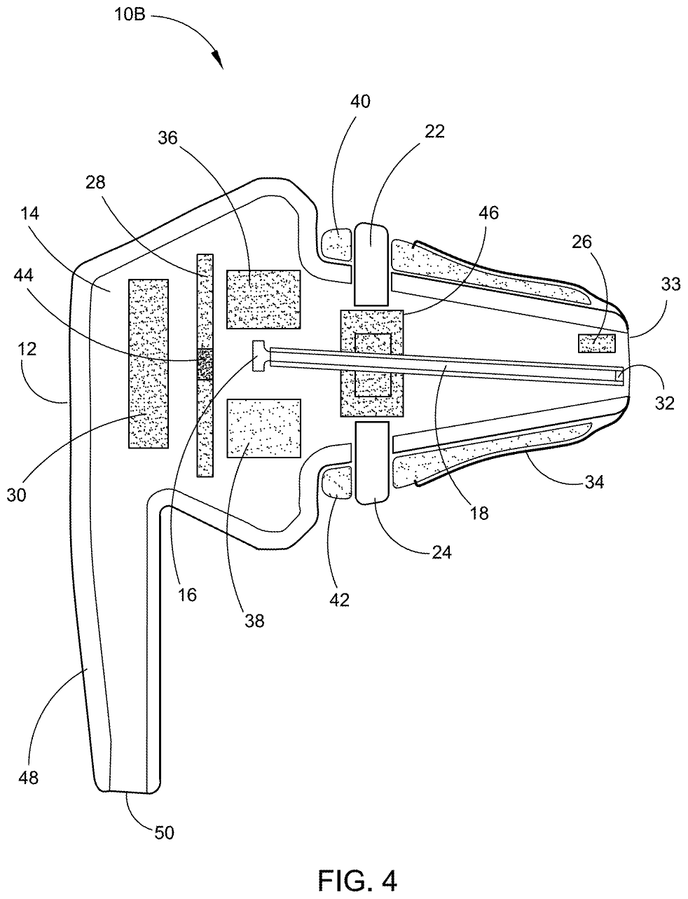

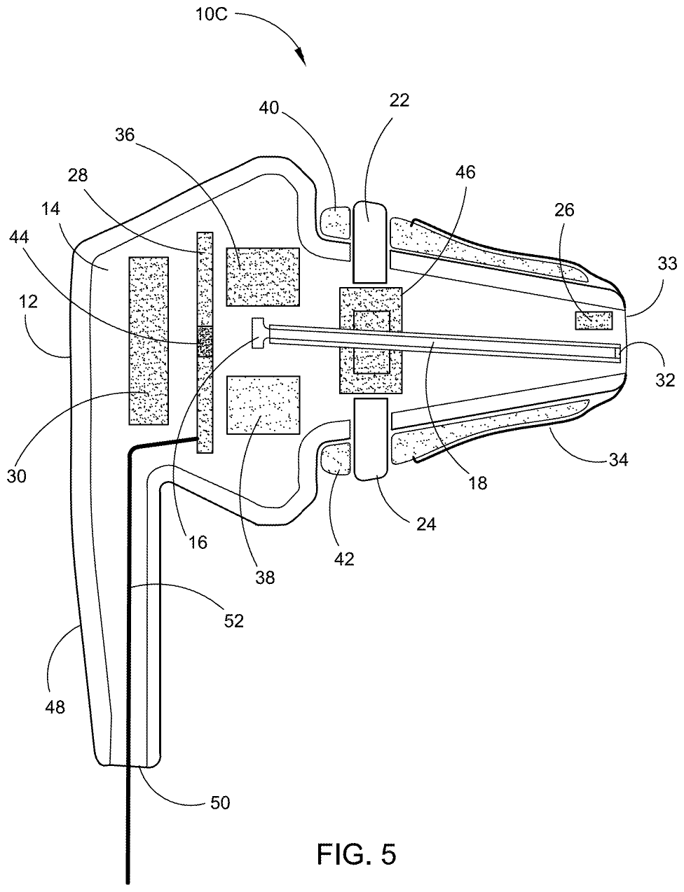

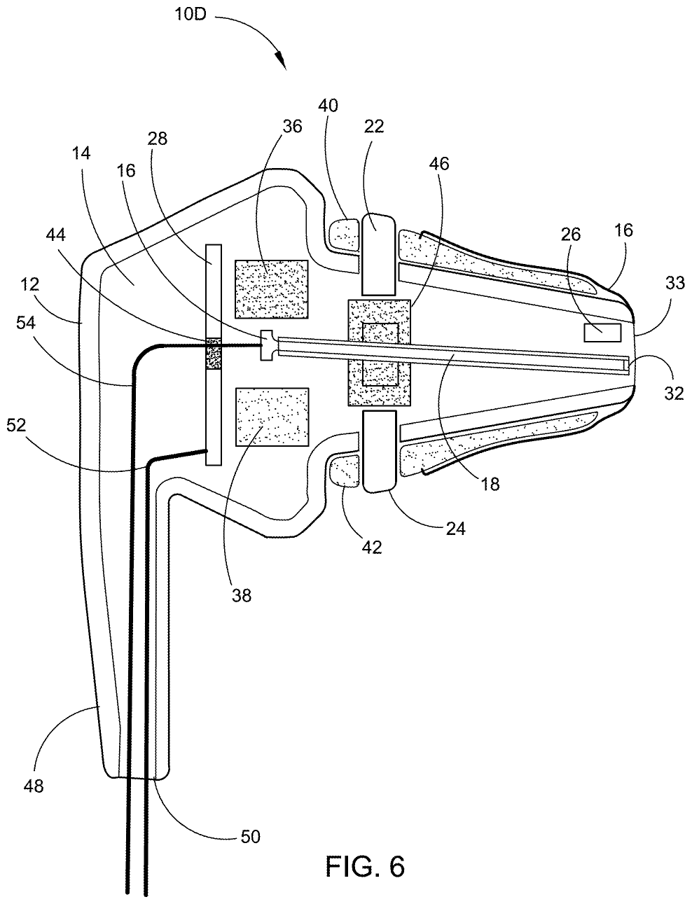

Referring now to FIG.'s 1, 4, 5, and 6, there are depicted a schematic of an exemplary photon source device 10A inserted into an ear canal of a subject as shown in FIG. 1, a cross-sectional view of a first embodiment PBMT device 10B as shown in FIG. 4, a second embodiment PBMT device 10C as shown in FIG. 5, and a third embodiment PBMT device 10D as shown in FIG. 6 of a photobiomodulation system photon source device (hereinafter photon source device) configured for use to deliver photobiomodulation therapy (hereinafter PBMT) to an ear of a subject. As shown in FIG. 4, a photon source device 10B comprises a housing 12 with an interior 14 which contains a plurality of components described in detail below. In embodiments, the housing 12 includes a form factor having fitting bio-interfaces 40 and 42 which is configured for insertion into an ear and/or an ear canal of the subject for PBMT operations, as may be used for treatment, prevention, diagnosis, evaluation of hearing loss, SNHL, tinnitus, ear ringing, or any combination thereof.

In embodiments, the photon source device 10B may be fitted to the subject, such that a distance between a light source 16 and the portion of the subject is controlled to optimize the safe and effective PBMT therapeutic light delivered. Because different subjects may have substantially different anatomies, optimal safe and effective PBMT therapy may require the photon source device 10A and 10B to be custom fitted to a particular anatomical structure of the subject. As a non-limiting example, if the PBMT system is used to diagnose, prevent, restore hearing loss and/or treat SNHL, then the photon source device 10B may be configured to be positioned within one or both ear canals of the subject for treatment of the middle ear, the inner ear, or both. Similarly, if the PBMT system is used to diagnose, prevent, and/or treat oral mucositis, then the photon source device 10B may be configured to be positioned within the mouth of the subject for treatment of the oral mucosal membranes. Accordingly, in embodiments of the PBMT system, the photon source device 10B may be custom fitted to an ear of the subject to deliver the therapeutic light to and through a tympanic membrane of the ear of the subject, or alternatively, may be custom fitted to a mouth of the subject to deliver the therapeutic light to a mucous membrane of the mouth of the subject. The photon source device 10B may utilize materials to custom fit to the patient's body location including coatings, disposable covers, malleable materials, and/or materials that are formed to fit the patient's specific body location, e.g., by a separate method.

In embodiments, the photon source device 10B may comprise a protective cover 34 which is seated about one or more fitted bio-interfaces 40 and 42. The protective cover 34 may include a forward aperture 33 on a forward end thereof, through which the therapeutic light from the light source 16 passes after beam formation at a light modulator 32. In embodiments, the forward aperture 44 may have a defined impact on the light signal, such as through one or more of attenuation and disbursement of the light signal. In addition, the protective cover 34 may be reusable or single-use, and in this manner, the photon source device 10B may be used by one subject only, or may be used by more than one subject without cross-contamination between subjects.

In embodiments, the photon source device 10B comprises a light source 16 configured to emit an optimal safe and effective quantity of the therapeutic light therefrom, a plurality of placement sensors 22 and 24 configured to detect that the photon source device 10B is positioned for use, and a control system 28 that operably connects a power source 30 to the light source 16. The photon source device 10B may be configured to emit one or more wavelength of light, e.g. red light, near-IR light, or both, among others; in embodiments, the photon source device 10B may be configured to emit light having one or more wavelengths including, but not necessarily limited to: 447 nm, 532 nm, 635 nm, 808 nm, and any combination thereof.

In embodiments, the plurality of positioning sensors 22 and 24 may be configured to detect whether the photon source device 10B is placed for use, and may conditionally emit one or more signals which communicate to the subject and/or another individual whether the device is correctly positioned. In embodiments, one or more placement sensors 22 and 24 may detect the location of the device inside the ear canal of the subject, and one or more proximity sensors 26 may detect a particular distance from the light source 16 to the portion of the subject's body to receive therapeutic light thereon, e.g., the tympanic membrane.

In embodiments, one or more placement sensors of the plurality of placement sensors 22 and 24 may emit an electronic signal, emit an audio signal, emit a visual signal, emit a haptic and/or tactile signal, complete an electronic circuit, or otherwise change or alter a state or a configuration of the photon source device 10B or the control system 28, when one or more of the sensors are activated. In embodiments, the one or more placement sensors 22 and 24 may be activated if the device is inserted into the ear canal, and the one or more proximity sensors 26 may be activated if the device is appropriately distanced from the portion of the subject's body to receive therapeutic light thereon. In embodiments, all sensors may need to be activated before using the photon source device 10B. In this manner, the photon source device 10B may be unable to be activated unless correctly positioned for use for the safe and effective delivery of therapeutic light.

In embodiments, the photon source device 10B may utilize a form of haptic feedback (e.g., kinesthetic communication) during one or more stages of operation, such as the photon source device 10B being properly placed, starting, delivering energy, working, stopping, and any combination thereof. In this manner, the subject, or another individual such as a caretaker, may operate the photon source device 10B using the sense of touch. In embodiments, the photon source device 10B may include one or more sensing means, of a plurality of sensing means, which measures noise exposure over time.

In embodiments, the plurality of placement sensors 22 and 24 may comprise a sensing means (e.g., a placement sensor) which detects proper placement of the photon source device 10B into an orifice (e.g., in an ear or in an ear canal) of a subject. In embodiments, the sensing means may be comprised of one or more suitable mechanisms, including but not necessarily limited to light and/or optical detection, detection of a mechanical change, detection of an electrical change, and/or detection of a galvanic skin response. In embodiments, the sensing means may need to be activated before the light source 16 is fully activated, to ensure the photon source device 10B is safely positioned for optimal therapeutic effect before use.

In embodiments, the sensing means may include, but may not necessarily be limited to, one or more skin color and skin condition sensors, one or more pulse oximetry SpO2 sensors, one or more StO2 sensors, one or more sensors capable of obtaining SmO2 measurements, one or more optical sensors, one or more optical imaging arrays, one or more heart rate (HR) sensors, one or more heart rate variability (HRV) sensors, one or more respiration rate sensors, one or more compression sensors, one or more electrodermal activity sensors (e.g., galvanic skin response (GSR) or galvanic skin conductance), one or more temperature sensors (e.g., skin, tympanic membrane), one or more sensors capable of measuring one or more neurological signals, one or more neural electrical impulse activity (EEG) sensors, and any combination thereof. In embodiments, the photon source device 10B may employ one or more sensing means to detect the presence of otitis media, cerumen (ear wax), other growths, foreign media, tympanic membrane surface/changes, ear canal topology, tympanic membrane topology or other conditions within the ear before, during, or after use of the invention.

In embodiments, the plurality of sensing means may comprise the proximity sensor 26 which automatically or upon command measures a distance between the light source 16 and the portion of the subject's body. In embodiments, the proximity sensor 26 may comprise a time-of-flight (TOF) sensor. The proximity sensor 26 may be operably connected to the control system 28, a control circuit of the photon source device 10B, or both, in order to enable safe and effective delivery of the therapeutic light to the portion of the subject's body.

In embodiments, the TOF sensor (e.g., 26) may include one or more components designed to determine the distance from the light source 16 to the portion of the subject to receive the PBMT treatment (e.g., skin, tympanic membrane). In embodiments, the TOF sensor may include any suitable optical, acoustic, or electromagnetic transmitter and receiver, or any combination thereof. In embodiments, the TOF sensor may include one or more photodetectors, photodetector arrays, microphones, antennae, and the like. One function of the TOF sensor is to detect if the photon source device 10B is properly positioned with the subject's body to deliver a safe and effective PBMT treatment prior to or during the treatment, and may operate inside the subject's ear, inside the subject's mouth, next to the subject's skin, or at another position within or adjacent to the subject. Another function of the TOF sensor is to detect if the photonic illumination plane orientation to deliver the optimal safe and effective PBMT.

In embodiments, the control system 28 may include an aperture 44 thereon, through which a component, such as an electrical wire, may pass which operably connects a power source 30 (e.g., a battery or a rechargeable battery) to the control system and/or light source 16. In this manner, the aperture 33 may facilitate insertion of an electronic or electrical component, or a portion thereof, therethrough, as may be needed during assembly of the photon source device 10B. In embodiments, the control system 28 may be operably connected to the power source 30 and the light source 16 and may be configured to control the photon source device 10B or an operation or method thereof. In embodiments, the light source 16 may be configured to deliver a safe and effective quantity of a therapeutic light to a portion of a subject, and to operate according to the control system 28.

In embodiments, the light source 16 may be comprised of one or more suitable sources of therapeutic light and may include one or more of any organic or inorganic light source. The light source 16 may be coherent, non-coherent, or both coherent and non-coherent. In embodiments, the light source 16 may be any suitable source of electromagnetic radiation, such as a light-emitting diode (LED), a laser, an incandescent light, a fluorescent light, a compact fluorescent light, one or more chemiluminescent compositions, one or more electrochemiluminescent compositions, a high-intensity discharge light, a halogen light, another suitable light source, or any combination thereof. In addition, it is contemplated herein that embodiments of the invention may be designed for reuse, and other embodiments of the invention may be designed for single use.

In embodiments, the photon source device 10B may be wearable by a subject, such that if the photon source device 10B is worn by the subject, the plurality of positioning sensors 22 and 24 detect that the photon source device 10B is positioned for optimal safe and effective use. In embodiments, the photon source device 10B may include any form factor suitable for its intended use, including but not necessarily limited to an ear insert form factor (e.g., as shown in FIG.'s 4, 5, and 6), a behind the ear form factor, an over-the-ear form factor, a mouthpiece form factor, a handheld form factor, a general form factor which may be used to treat any part of the body, and the like. In this manner, the photon source device 10B may be worn by the subject while the subject performs other tasks, and therapy may be delivered on a constant or regular basis throughout a period.

In embodiments, the photon source device 10B may also comprise a light modulator 32. The light modulator 32 may be configured to convert a first wavelength of light into one or more second or additional wavelengths of light to produce the optimal safe and effective PBMT for the physiological state of the subject. In embodiments, any suitable optical mechanism for modulating light may be utilized for the light modulator 32, including but not necessarily limited to one or more filters, one or more waveguides, one or more quantum dots, one or more lenses, and any combination thereof. In this manner, the photon source device 10B may be configured to deliver an optimal safe and effective PBMT in a particular treatment mode of operation of the device.

In embodiments, the photon source device 10B may comprise a control system 28 operably connected to the photon source device 10B, and the control system 28 may be partially or completely integral with the photon source device 10B. In embodiments, the control system 28 may be configured to control at least part of the photon source device 10B. The control system 28 may include computer hardware and software elements to enable partially and/or fully automated control of the photon source device 10B, as may be desired to perform one or more methods of the invention. In embodiments in which the control system 28 is partially integrated with the photon source device 10B, some portion of the control system 28 may reside on or with the photon source device 10B and some other portion of the control system 28 may reside on or with another device, such as a personal computing device (e.g., smartphone, smart watch, computer), or a networked computer system, e.g., as may be utilized as part of a treatment service. In embodiments having full integration of the control system 28 with the photon source device 10B, the entire control system 28 may reside on or with the photon source device 10B, and in this manner, the control system 28 may be fully integrated with the photon source device 10B for localized control of the device during use. In embodiments, the control system may be configured to enable local control, remote control, or both local and remote control, according to a particular implementation. In embodiments, one or more operable connections between the control system 28 (or a component thereof) and the photon source device 10B may be wired, wireless, or any combination thereof. In embodiments in which the control system 28 is in wireless communication to the photon source device 10B, a split control system 28 may be utilized, wherein part of the control system 28 is local and part of the control system 28 is remote. In embodiments, one or more wireless connections of the invention may include one or more optical connections, one or more radiofrequency (RF) connections, one or more acoustic connections, one or more Wi-Fi connections, one or more Bluetooth® connections, one or more cellular connections, or any combination thereof.

In embodiments, the photon source device 10B may be capable of sensing and generating acoustic frequencies with an in-ear microphone/receiver 36 and speaker 38, or one or more similar devices. The microphone/receiver 36 and speaker 38 may be utilized to detect auditory acuity changes (e.g., gains and losses) over time, including changes in the ability to hear different sound frequencies as well as different sound intensities the subject is exposed to during a period of time. Along with the other components of the system, the microphone/receiver 36 and speaker 38 provide data to determine the physiological state of the subject, such as auditory acuity, and/or to detect total ambient sound exposure during use and/or during the time between applications of the PBMT therapy, for example. Accordingly, the microphone/receiver 36 and speaker 38 may be present in embodiments for which the intended use is diagnosis, evaluation, treatment, restoration and/or prevention of hearing loss, particularly sensorineural hearing loss, tinnitus and/or ear ringing.

In embodiments, the photon source device 10B may be integral with another device, such as a device that protects the ear from excessive sounds and/or reduces ambient sound (e.g. industrial/military protective headphones, noise cancellation headphones). In embodiments, the photon source device 10B may cooperate with the protective headphones to deliver PBMT to prevent and/or restore hearing loss, SNHL, tinnitus, and/or ear ringing. In this manner, through the use of such a combination, a subject wearing a combinatorial device may experience lower environmental noise and have a lower risk for hearing loss, and may also receive PBMT to prevent or treat hearing loss, SNHL, tinnitus, or ear ringing. In embodiments, the combinatorial device may sense noise exposure directly, or receive input from another device (e.g., a computer, a cellular phone, etc.) communicating noise exposure to form a singular and/or a cumulative data set for noise exposure; such a singular and/or cumulative data set may be utilized by the combinatorial device to determine the optimal safe and effective therapy which may be required by the subject for a period of time, e.g., daily, weekly, monthly, or some other period of time.

In embodiments, a waveguide 18 is positioned between the light source 16 and the light modulator 32. The waveguide 18 may be any structure that guides light waves from the light source 16 to the light modulator 32 with minimal loss of energy by optimizing the delivery of light energy to the subject. The waveguide 18 may be necessary to maintain and/or define the amount of light delivered to the light modulator 32, and in this manner, a defined quantity of light may be available for modulation by the light modulator 32 prior to illuminating the selected subject's body location.

In embodiments, a light source aimer and/or collimating feature 46 may be included in the photon source device. The light source aimer and/or collimating feature 46 may be any suitable structure for adjusting one or more angles of one or more of the light sources 16, the waveguide 18, and the light modulator 32. Because different subjects have different anatomical shapes of ear canals, one or more angles of the waveguide 18 may need to direct the optimal therapeutic light from the light source 16 toward the selected tissue, e.g. tympanic membrane, cochlea, etc. Exemplary angles that may be adjusted by the light source aimer and/or collimating feature 46 include an angle about a vertical axis, an angle about a horizontal axis, and any combination thereof.

Now referring to FIG. 5 and FIG. 6, in embodiments, the photon source device 10D includes a stem 48 which may be hollow and include a stem aperture 50 on a lower portion thereof. The stem 48 may be sized to enable one or more wires and/or fiberoptic cables to pass therethrough. Exemplary wires which may pass through the stem 48 and the stem aperture 50 include a cable 54 carrying one or more wave guides transmitting light from an external light source, a wire such as a control wire 52 carrying electrical power from an external power source, a wire from a partial of completely external control system, and any combination thereof. In the embodiment of FIG. 6, the photon source device 10D may be a self-contained earphone embodiment, and in such an embodiment, the stem 48 and/or the stem aperture 50 may be omitted from a particular design as needed. In the embodiment of FIG. 5, the photon source device 10C may be a semi-contained earphone embodiment, wherein control of the photon source device 10C is achieved through an external control mechanism and/or wherein power is delivered to the control system 28 by the control wire 52. In the embodiment of FIG. 6, the photon source device 10D may be an externally-controlled earphone embodiment, wherein control and power are relayed by the control wire 52 and wherein light is delivered from the external light source by the cable 54, which may be a fiberoptic cable.

In embodiments, the control system 28 may comprise a non-transitory computer-readable storage medium with instructions encoded thereon which, when executed by a processor, causes a PBMT system which comprises the photon source device 10A-10D to perform all or part of a method of the invention. In embodiments, the method may be wholly or partially performed by the control system 28 and the photon source device 10A-10D. In this manner, the method may be completely performed by a system of the invention, or alternately, may be partially performed by the system of the invention.

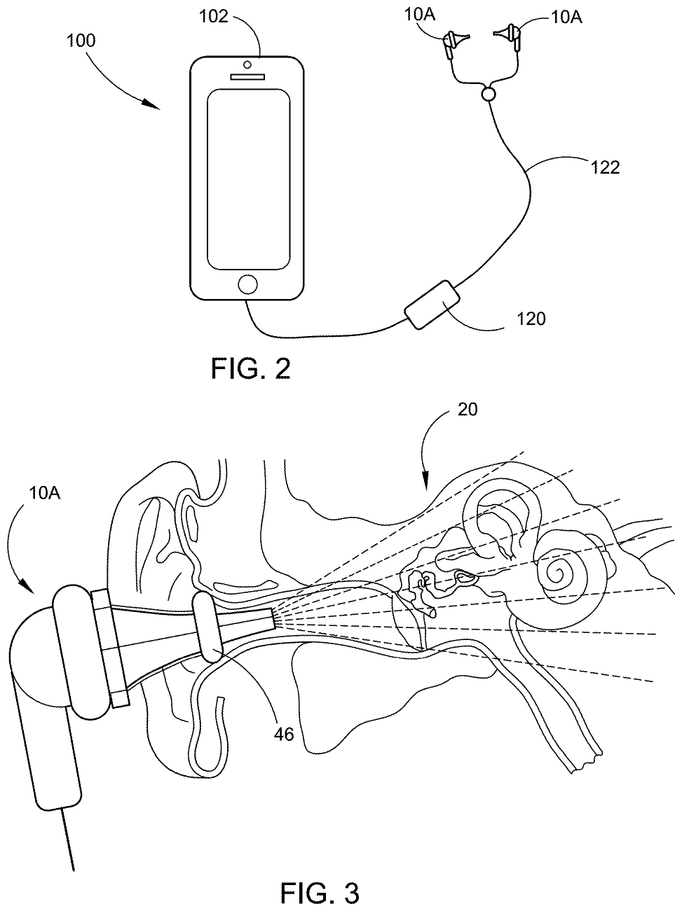

FIG. 2 depicts a combination photobiomodulation/smartphone device 100 configured in a dual photon source device 10A for insertion into one or both of a subject's ears, including a control feature/light source 120 in communication with a smartphone 102 or like device, both of which are connected to the two photobiomodulation devices via a wired connection 122 (see FIG. 5 below).

FIG. 3 depicts a detailed cross-sectional view of a subject's ear anatomy with a photobiomodulation system photon source device 10A inserted into the ear canal illustrating the illumination of therapeutic light 20 into the subject's tympanic membrane, middle ear, cochlea and inner ear.

Referring now to FIG. 2 and FIG. 3, there are depicted an illustration of an exemplary PBMT combination photobiomodulation/smartphone system 100 as shown in FIG. 2, and an illustration of the exemplary PBMT system photon source device 10A in use to diagnose, prevent, restore hearing loss and/or treat a hearing condition as shown in FIG. 3. In embodiments, a computer system 102 (e.g., one or more of a personal computer, a tablet, a cellular phone, a smartphone, and the like) is operably connected to one or more photon source devices 10A via connection 122. In embodiments, connection 122 may be a wired connection, a wireless connection, an RF connection, an audio connection, an optical connection, or any other connection type or combination thereof. In embodiments, a control feature 120 (which may comprise all or part of the control system) may be included to enable full or partial control of the photon source device 10A and/or a control system of the invention. To use the photon source device 10A to diagnose, prevent, restore hearing loss and/or treat a hearing condition, the photon source device 10A is inserted into one or both ear canals of a subject, as shown in FIG. 3. Upon inserting the photon source device 10A into the ear canal, one or more sensors of a plurality of sensors may be triggered to enable the photon source device 10A to be activated, as described elsewhere herein. In this manner, the photon source device 10A may be activatable if correctly positioned for use.

FIG. 4 depicts an enlarged cross-sectional detailed view of a photobiomodulation device 10B which operates wirelessly and is powered by an on-board battery power source 30, and is discussed in greater detail above.

FIG. 5 depicts an enlarged cross-sectional detailed view of a photobiomodulation device 10C which operates wirelessly and is powered by an on-board battery 30 as well as an external wired power/control source wire 52, and is discussed in greater detail above.

FIG. 6 depicts an enlarged cross-sectional detailed view of a photobiomodulation device 10D which operates wirelessly, has an external light source connection, and is powered by an on-board battery 30 as well as an external wired power/control source wire 52, and a fiber optic light source 54, and is discussed in greater detail above.

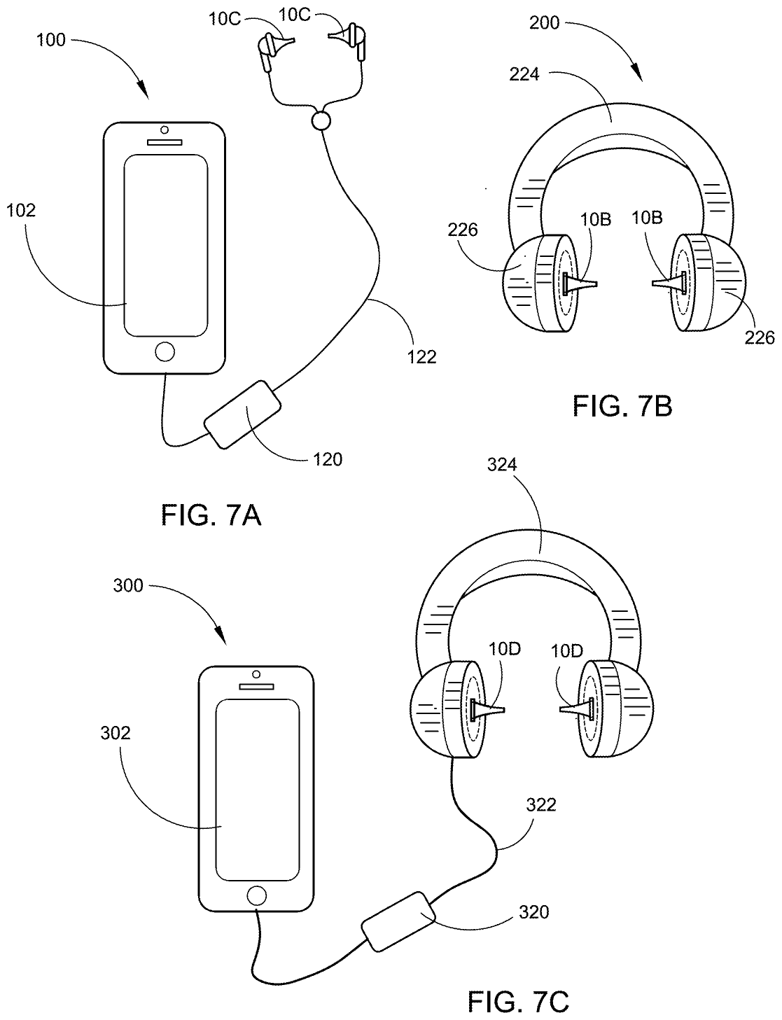

FIG. 7A depicts a combination photobiomodulation/smartphone system 100 photobiomodulation device 10C configured in a dual device for insertion into one or both of a subject's ears, including a light source 120 in communication with a smartphone or like device via a wired connection 122, both of which are connected to the two photobiomodulation devices 10C.

FIG. 7B depicts a combination photobiomodulation/headset system 200 configured in a head set 224 style dual devices 10B for insertion into both of a subject's ears, including a self-contained control feature/light source 226 in wireless communication with a smartphone or like device.

FIG. 7C depicts a combination photobiomodulation/smartphone/headset system 300 configured in a head set 324 style dual device 10D for insertion into both of a subject's ears including a light source 320 in communication with a smartphone 302 or like device, both of which are wire 322 connected to the two photobiomodulation devices.

Referring now to FIG.'s 7A, FIG. 7B, and FIG. 7C, there are depicted illustrations of a first (FIG. 7A), a second (FIG. 7B), and a third (FIG. 7C) exemplary combination PBMT systems according to the present invention. In embodiments, the PBMT system may include an “ear bud” form factor 10A-10D, configured to be inserted into the ear canal of the subject (FIG. 7A). In embodiments, the photon source device(s) 10A-10D may be operably connected to the smartphone/computer system 102 via connection 122, which may be a wired connection, a wireless connection, an RF connection, an audio connection, an optical connection, or any other connection type or combination thereof. In embodiments, a control feature/light source 120 (which may comprise all or part of the control system) may be included to enable fill or partial control of the photon source device 10A-10D and/or a control system of the invention. In embodiments, the PBMT system 200 may include an “over-ear” form factor 224, configured to both cover the ear and to be inserted into the ear canal of the subject (FIG. 7B); in such embodiments 200, an over-ear portion 224 may include an integral control feature 226, and may be configured for noise cancellation and/or acoustical acuity therapy using the photon source device(s) 10A-10D and/or another component of the PBMT system (FIG. 7B). In embodiments, the PBMT system 300 may include an “over-ear” form factor 324 with an external control feature 320 (FIG. 7C); in such embodiments, the over-ear potion 324 may include the photon source device(s) 10D operably connected to the computer system 302 via a wire connection 322. Selection of one or more particular embodiments may be driven by cost and/or design considerations.

In embodiments, the invention provides a method for photobiomodulation, comprising: evaluating a physiological state of a subject and compiling a first signature from data of the subject's profile, first evaluation, positioning a photon source device within and/or adjacent to the subject, activating the photon source device to deliver a safe and effective quantity of a therapeutic light to a portion of the subject, evaluating the physiological state of the subject and compiling a second signature from data of the second evaluation, and comparing the first signature with the second signature to determine change or the probability of change in the physiological state. The method may be performed by the control system and the photon source device, an individual such as a healthcare worker, the subject, or any combination thereof, regardless of whether the control system is local to the photon source device, remote to the photon source device, or both local and remote. The physiological state may correspond to a condition, disease, or disorder for which treatment with PBMT is being applied. For example, if PBMT is being used to treat SNHL, the physiological state may include hearing sensitivity, auditory acuity, medical history, or any combination thereof.

In embodiments, evaluation of the physiological state may be performed by the subject, an individual such as a healthcare worker, an authorized third party or any combination thereof. The physiological state may correspond to a condition, disease, and/or disorder for which treatment with PBMT is being applied. For example, if PBMT is being used to treat SNHL, the physiological state may include current hearing sensitivity, past auditory sensitivity, current auditory acuity, past auditory acuity, or any combination thereof. Additional physiological states which may be utilized by the present invention include, but are not limited to, physiological sensing (e.g. heart rate, heart rate variability, electro cardiogram, pulse wave velocity, blood flow, blood pressure, skin/tissue/core temperature, skin color, skin topology, pulse oximetry, tissue oximetry, tissue composition, tissue impedance, electroencephalogram, evoked potential voltages, galvanic skin response/skin conductance, body motion, body position, respiratory rate, respiratory volume, respiratory noise, VO₂ max, algorithmically transformations of one or more of these physiological parameters into a different bioparameter), blood tests for stress and inflammatory biomarkers, genetic tests, microbiome tests, auditory tests, time since last PBMT treatment, number of previous PBMT treatments, last PBMT treatment dose energy e.g. J/cm², and/or similar dose measurements, type of prior PBMT treatment, subject age, subject gender, subject ethnicity, and subject medical history (including but not necessarily limited to injuries such as punctured and/or ruptured tympanic membrane, procedures, prescriptions including current and prior prescriptions, presence of antibiotics or steroids or ototoxic compositions, audiometry including auditory acuity loss, range, and length of time since last PBMT treatment, co-morbidities, and the like). Additional data may also be included such as the amount of acoustical energy that the subject has been exposed to for a period of time.

In embodiments, the method may further comprise adjusting the quantity of thetherapeutic light, adjusting a size of the portion of the subject's body which is illuminated/receives the therapeutic light, adjusting the sequence of therapeutic light applied, adjusting the pattern in which the therapeutic light is applied, or any combination thereof. The adjustment may be made by the control system, by the subject, by the individual such asthe healthcare worker, or any combination thereof, regardless of whether the control systemis local to the photon source device, remote to the photon source device, or both local and remote. In this manner, the treatment may be adjusted inside the medical setting and outsidethe medical setting as needed.

In embodiments, the method may further comprise administering one or more exogenous material and/or treatments to the subject. Exemplary exogenous materials include treatments such as localized and/or systemic therapies, including but not limited to pharmaceutical compositions, biological compositions, supplements, cell-based therapies, and other therapeutic services. In embodiments, the exogenous material may comprise a stern cell. A variety of factors may limit the effectiveness of stem cell therapy, and the method of the present invention may be used to overcome some or all of these factors to improve the safety and efficacy of a combined PBMT and stem cell therapy.

In embodiments, an output of the method of the invention includes recommendations for, and/or adjustments to, a dosing protocol adjustment for current and subsequent treatment applications (e.g., treatment duration, light frequency, light sequence, light intensity, and/or light pattern), which may be performed by a clinician, an audiologist, a patient, a care provider, or any combination thereof. The method may evaluate treatment efficacy and/or progress and escalate the intervention to a clinician if the subject is not achieving the requisite progress with the treatments on their own. The method may also output the results of sensor physiological and/or gains or losses in auditory sound frequency, intensity or degradation, recovery, and/or homeostasis. The auditory tests output may also determine the subject's ability to hear words and/or digits transmitted by the system. The method may output correlations between treatments, patient information, and auditory acuity by combining and evaluating data sets from one or more patients. In addition, the method may utilize artificial intelligence (AI) or machine learning (ML) analysis, which may include one or more of predicted future auditory acuity loss and/or restoration, risk profile and index of future auditory acuity loss and/or restoration, combine data from other subjects to increase accuracy of prediction and risk profile, combine data from other subjects to improve treatment profile such as duration, light wavelength, type (combination of photonic with other energy, supplements, drugs, foods, lifestyle, and the like), and any combination thereof.

In embodiments, the method of the control system may further comprise adjusting the quantity of the therapeutic light, wavelength, wavelength illumination sequence, wavelength illumination pattern, the area of the subject's body which receives the therapeutic light to optimize the PBMT, or any combination thereof. In embodiments, the quantity adjusted may include all wavelengths of the therapeutic light or a subset of wavelengths thereof. For example, if deeper penetration of tissue is needed, the wavelength of therapeutic light delivered to the portion of the subject may include a greater intensity of one or more longer wavelengths, optionally combined with a lesser intensity of shorter wavelengths. The sequence of light wavelengths emitting therapeutic light may be set to emit one or more wavelengths simultaneously, sequentially, in a graduated overlap, and/or in one or more patterns, or any combination of those elements. Similarly, if a greater portion of the subject needs to be irradiated with the safe and effective PBMT to facilitate treatment, then the optical properties of the photon source device may be adjusted to irradiate a larger area of the portion of the subject.

Combination therapies

Generally, the present invention provides an improved localized PBMT therapy for the effective treatment and management of conditions, diseases, and disorders, which may be combined with any other known or unknown treatment, primary or secondary (adjunctive) treatment, localized or systemic treatment, or any combination thereof, whether intended for the same or a different condition, disease, or disorder. Exemplary therapies which may be combined with PBMT therapy of the present invention include, but are not limited to, biologic therapies, device therapies, drug therapies, gene therapies, service therapies, and supplement therapies.

Biologic therapies

In embodiments, PBMT may be combined with one or more anti-apoptosis and/or anti-necrosis biologic therapeutics. As a non-limiting example, PBMT may be combined with a JNK inhibitor such as AM-111 (Sonsuvi®) or similar, XG-102 (brimapitide) or similar, or any combination thereof. In this manner, the therapeutic action of PBMT may benefit from or be enhanced by one or more anti-apoptosis and/or anti-necrosis biologic therapeutics.

In embodiments, PBMT may be combined with one or more antioxidant enzymatic scavenger biologic therapeutics. As a non-limiting example, PBMT may be combined with an anti-oxidant such as superoxide dismutase, catalase, glutathione peroxidases, thioredoxin peroxiredoxin, glutathione transferase, or any combination thereof. In this manner, the therapeutic action of PBMT may benefit from and/or be enhanced by one or more antioxidant enzymatic scavenger biologic therapeutics.

In embodiments, PBMT may be combined with one or more cell growth stimulating biologic therapeutics. As a non-limiting example, PBMT may be combined with a cell growth stimulator such as epidermal growth factor (EGF), a gamma secretase inhibitor, a WNT agonist, brain-derived neurotrophic factor (BDNF), an anti-NOTCH antibody, a composition comprising one or more progenitor and/or stem such as umbilical cord blood, a modulator of a stem cell signaling pathway such as one or more of Wnt, Notch, and Sonic Hedgehog, signaling pathways for development of hair cells from stem cells, one or more other exogenous factors which promote the expression of Math1 transcription factor, a composition comprising one or more mesenchymal stem cells (MSC), a composition comprising one or more pillar and/or Deiter cells, bone marrow, bone marrow-derived mesenchymal stern cells (MSCs), or any combination thereof. In this manner, the therapeutic action of PBMT may benefit from or be enhanced by one or more cell growth stimulating biologic therapeutics.

In embodiments, PBMT may be combined with one or more cell growth regulator biologic therapeutics. As a non-limiting example, PBMT may be combined with one or more bone remodeling modulators, such as sclerostin (bone growth modulator through Wnt inhibition). In this manner, the therapeutic action of PBMT may benefit from and/or be enhanced by one or more cell growth regulators.

In embodiments, PBMT may be combined with one or more cell growth stimulating biologic therapeutics. As a non-limiting example, PBMT may be combined with one or more LATS kinase compositions, stimulators, or inhibitors, such as one or more gene therapies which deliver, stimulate, or inhibit LATS kinase, or any combination thereof. In this manner, the therapeutic action of PBMT may benefit from and/or be enhanced by one or more cell growth stimulating biologic therapeutics.

In embodiments, PBMT may be combined with a biologic/drug combination for enhanced drug delivery. As a non-limiting example, PBMT may be combined with a therapeutic such as an auris pressure modulator, a combination of one or more of a immunomodulatory agent, an interferon, a channel modulator, a gamma-globulin, a chemotherapeutic agent, an anti-viral, an antibiotic, an anti-vascular agent, or any combination thereof. In embodiments, the biologic/drug combination may comprise a gamma secretase modulator and/or a pharmaceutically acceptable prodrug or salt thereof, and about 15% to about 35% by weight of a polyoxyethylene-polyoxypropylene triblock copolymer. In this manner, the therapeutic action of PBMT may benefit from and/or be enhanced by a biologic/drug combination for enhanced drug delivery.

Device therapies

In embodiments, PBMT may be combined with one or more acoustical energy therapies. As a non-limiting example, PBMT may be combined with acoustical energy that downregulates and/or inhibits detrimental physiological changes that are associated with, correlated with, or causative of sensorineural auditory acuity (frequency and/or intensity) loss and/or tinnitus. In the alternative or in addition, PBMT may be combined with acoustical energy that upregulates and/or stimulates beneficial physiological changes that are associated with, correlated with, and/or causative of sensorineural hearing acuity (frequency and/or intensity) loss and/or tinnitus. In this manner, the therapeutic action of PBMT may benefit from and/or be enhanced by one or more acoustical energy therapies.

In embodiments, PBMT may be combined with one or more electromagnetic and/or electrical therapies. As a non-limiting example, PBMT may be combined with an electrical stimulation which promotes neural plasticity changes, such as an electrical therapy that downregulates and/or inhibits detrimental physiological changes that are associated with, correlated with, or causative of sensorineural auditory acuity (frequency and/or intensity) loss and/or tinnitus. In the alternative or in addition, PBMT may be combined with an electrical stimulation which promotes neural plasticity changes, such as an electrical therapy that upregulates and/or stimulates beneficial physiological changes that are associated with, correlated with, and/or causative of sensorineural auditory acuity (frequency and/or intensity) loss and/or tinnitus. In this manner, the therapeutic action of PBMT may benefit from and/or be enhanced by one or more electromagnetic or electrical therapies.

In embodiments, PBMT may be combined with one or more device therapies which enhance drug delivery. As a non-limiting example, PBMT may be combined with one or more therapies such as such as electrophoresis (opens pores to allow delivery of exogenous drugs, biologics, cellular treatments) which may be electrical or photonic, iontophoresis, reverse iontophoresis, or any combination thereof. In this manner, the therapeutic action of PBMT may benefit from and/or be enhanced by one or more device therapies which enhance drug delivery.

Drug therapies

In embodiments, treatment with other drug therapies and/or exposure to ototoxic chemicals, metals and asphyxiants may cause hearing loss which requires treatment with PBMT of the present invention. As a non-limiting example, treatment with cytotoxic agents (e.g., antibiotics such as aminoglycosides), chemotherapeutic agents (e.g., carboplatin, cisplatin) may cause hearing loss. In addition, treatment with antibiotics (e.g., ciprofloxacin) and/or aminoglycosides (e.g., gentamicin, streptomycin), and ciprofloxacin may cause hearing loss. Accordingly, in embodiments, PBMT may be combined with one or more of these treatments to mitigate, prevent or treat hearing loss, SNHL, tinnitus, or ear ringing in a particular subject. Ototoxic chemicals may cause hearing loss such as: solvents e.g. carbon disulfide, n-hexane, toluene, p-xylene, ethylbenzene, n-propylbenzene, styrene and methylstyrene, trichloroethylene; asphyxiants e.g. carbon monoxide, hydrogen cyanide and its salts, tobacco smoke; nitriles e.g. 3-Butenenitrile, cis-2-pentenenitrile, acrylonitrile, cis-crotononitrile, 3,3′-iminodipropionitrile. Ototoxic metals and compounds may cause hearing loss e.g. mercury compounds, germanium dioxide, organic tin compounds, lead. Accordingly, in embodiments, PBMT may be combined with one or more other treatments defined herein to mitigate, prevent, restore and/or treat hearing loss, SNHL, tinnitus, or ear ringing in a subject caused by ototoxic chemicals.

In embodiments, PBMT may be combined with one or more anti-apoptotic or anti-inflammatory drug therapies. As a non-limiting example, PBMT may be combined with a therapeutic such as an inhibitor of BCL-2, an inhibitor of glycogen synthase kinase 3 (GSK3β), or any combination thereof. In this manner, the therapeutic action of PBMT may benefit from and/or be enhanced by one or more anti-apoptotic drug therapies.

In embodiments, PBMT may be combined with an anti-coagulant therapy. As anon-limiting example, PBMT may be combined with an anti-coagulant such as ancrod. In thismanner, the therapeutic action of PBMT may benefit patients receiving an anti-coagulant therapy.

In embodiments, PBMT may be combined with one or more anti- inflammatory therapeutic drugs. As a non-limiting example, PBMT may be combined with one or more anti-inflammatory agents such as an antagonist of IL-1 receptor (e.g., anakinra), methotrexate, a therapy that increases adenosine signaling, a steroid (e.g., dexamethasone, corticosteroid, glucocorticoid, mineralocorticoid, anakinra), an anti-TNF-α agent, SPI-1005, Ebselen, or any combination thereof. In this manner, the therapeutic action of PBMT may benefit from and/or be enhanced by one or more anti-inflammatory therapeutic drugs.

In embodiments, PBMT may be combined with one or more anti-oxidant therapeutic drugs. As a non-limiting example, PBMT may be combined with an anti-oxidant such as sodium thiosulfate, EPI-743, vatiquinone, glutathione, a histone deacetylase inhibitor, a pan-HDAC inhibitor (e.g., SAHA), or any combination thereof. In this manner, the therapeutic action of PBMT may benefit from and/or be enhanced by one or more anti-oxidant therapeutic drugs.

In embodiments, PBMT may be combined with an anti-oxidant and/or free radical scavenger. As a non-limiting example, PBMT may be combined with a therapeutic such as HPN-07 (4-[(tert-butylimino)methyl] benzene-1, 3-disulfonate N-oxide) (disufenton sodium),In this manner, the therapeutic action of PBMT may benefit from and/or be enhanced by an anti-oxidant and/or free radical scavenger.

In embodiments, PBMT may be combined with an anti-viral agent. As a non-limiting example, PBMT may be combined with an anti-viral therapy such as valgancilovir, which is used to treat cytomegalovirus (CMV) infection which may lead to hearing loss. In this manner, the therapeutic action of PBMT may benefit patients receiving anti-viral therapy.

In embodiments, PBMT may be combined with a channel modulator drug therapeutic. As a non-limiting example, PBMT may be combined with a channel modulator such as such as AUT00063 or zonisamide. In this manner, the therapeutic action of PBMT may benefit from and/or be enhanced by a channel modulator drug therapeutic.

In embodiments, PBMT may be combined with a channel modulator or glutamate signaling drug therapeutic. As a non-limiting example, PBMT may be combined with a drug therapeutic such as gacyclidine, one or more N-methyl-D-aspartate (NMDA) receptor antagonists, or any combination thereof. In this manner, the therapeutic action of PBMT may benefit from and/or be enhanced by a channel modulator and/or glutamate signaling drug therapeutic.

In embodiments, PBMT may be combined with a channel modulator and/or neurotransmission modulator. As a non-limiting example, PBMT may be combined with a channel modulator and/or neurotransmission modulator drug therapeutic such as such as Zonisamide. In this manner, the therapeutic action of PBMT may benefit from or be enhancedby a channel modulator and/or neurotransmission modulator.

In embodiments, PBMT may be combined with one or more channel modulators and/or neurotrophic growth factors. As a non-limiting example, PBMT may be combined with a drug therapeutic such as a central nervous system (CNS) modulator, such asAUT00063, BDNF, or any combination thereof. In this manner, the therapeutic action of PBMT may benefit from and/or be enhanced by one or more channel modulators and/or neurotrophic growth factors.

In embodiments, PBMT may be combined with one or more neurotransmission modulators. As a non-limiting example, PBMT may be combined with a neurotransmission modulator such as PF-04958242 (α-amino-3-hydroxy-5-methyl-4-isoxazolepropionic acid potentiator), R-azasetron besylate (5-HT3 receptor antagonist and calcineurin inhibitor), vestipitant (NK1 receptor selective antagonist), or any combination thereof. In this manner, the therapeutic action of PBMT may benefit from and/or be enhanced by one or more neurotransmission modulators.

Gene therapies

In embodiments, PBMT may be combined with one or more cell growth stimulator gene therapies. As a non-limiting example, PBMT may be combined with a cell growth stimulator drug gene therapy such as CGF166 (adenovirus vector containing cDNA for the human Atonal transcription factor (Hath1)), one or more AAV gene therapies (e.g., delivery of Atoh1, VGLUT3, or both), or any combination thereof (Atoh1 may also be referred to as Math1 (mouse) and/or HATH1 (human)). In this manner, the therapeutic action of PBMT may benefit from and/or be enhanced by one or more cell growth stimulator gene therapies.

In embodiments, PBMT may be combined with gene therapy containing any suitable gene for delivery which utilizes a particular vehicle for delivery. As a non-limiting example, PBMT may be combined with a therapy comprising a suitable gene therapy delivery vehicle such as AAV-i.e. which is an AAV viral vector with select peptides inserted to make it optimal for delivery into the inner ear. In this manner, the therapeutic action of PBMT may benefit from and/or be enhanced by gene therapy containing any suitable gene for delivery which utilizes a particular vehicle for delivery.

In embodiments, PBMT may be combined with one or more cell growth stimulator biologic and/or gene therapeutic approaches. As a non-limiting example, PBMT may be combined with cochlear hair cell regeneration therapy such as promoting ATOH1 expression, blocking NOTCH activity (e.g., using one or more gamma-secretase inhibitors), orany combination thereof. In this manner, the therapeutic action of PBMT may benefit from and/or be enhanced by one or more cell growth stimulator biologic and/or gene therapeutic approaches.

In embodiments, PBMT may be combined with one or more cell growth regulator gene therapies. As a non-limiting example, PBMT may be combined with a gene therapy such as delivery of p27Kip1, which must be tightly regulated to prevent overgrowth and/or lack of hair cell formation (both of which lead to hearing loss). In this manner, the therapeutic action of PBMT may benefit from and/or be enhanced by one or more cell growth regulator gene therapies.

Service therapies

In embodiments, PBMT may be combined with one or more service therapies which benefit physiological, neural, and cognitive parameters of the subject. As a non-limiting example, PBMT may be combined with one or more service therapies such as acupuncture, cognitive behavior therapy, coping skills, physical activities, exercise, food, meditation, sleep treatments, stress treatments, yoga, stellate ganglion blocking (SGB), or any combination thereof. In this manner, the therapeutic action of PBMT may benefit from and/or be enhanced by one or more service therapies which benefit physiological, neural, and cognitive parameters of the subject.

Supplement therapies

In embodiments, PBMT may be combined with one or more anti-oxidant and/or anti-inflammatory supplement therapies. As a non-limiting example, PBMT may be combined with one or more supplement therapies such as vitamin A (trans retinol 2), vitamin C(ascorbic acid), vitamin E (tocopherol and tocotrienols e.g., alpha tocopherol), beta carotene, glutathione, D-methionine, N-acetylcysteine, glutathione peroxidase mimicry (e.g., Ebselen), sodium thiosulfate, alpha lipoic acid, HPN-07 cofactor of mitochondrial enzymes, turmeric, a free radical scavenger, zinc gluconate, and any combination thereof. In this manner, the therapeutic action of PBMT may benefit from and/or be enhanced by one or more anti-oxidant and/or anti-inflammatory supplement therapies.

Assorted therapies

Assorted therapies that may be combined with PBMT therapy include, butare but not limited to, treatment with one or more anti-inflammatory agents, treatment with one or more beneficial supplements, or any combination thereof.

In embodiments, the portion of the subject which receives PBMT may also comprise an exogenous material. Exemplary exogenous materials include treatments such as localized and/or systemic therapies, including but not limited to pharmaceutical compositions, biological compositions and/or cell-based therapies. In embodiments, the exogenous material may comprise a stem cell. A variety of factors may limit the effectiveness of stem cell therapy, and the PBMT system of the present invention may be used to overcome these factors to improve the effectiveness of stem cell therapy. In embodiments, PBMT may increase the stem cell efficacy by causing the stem cells to preferentially become hair cells by exposing the stem cells to a PBMT protocol that may include one or more light wavelengths and therapeutic emission sequences and patterns. In embodiments, PBMT may be combined with one or more intratympanic injections of one or more stem cells.

Additional assorted therapies that may be combined with PBMT include one or more anti-TNF-α agents, one or more auris pressure modulators, one or more CNS modulators, one or more cytotoxic agents, one or more anti-apoptotic agents, one or more bone-remodeling modulators, one or more free radical modulators, one or more ion channel modulators, one or more antibiotics (e.g., ciprofloxacin), one or more steroids (e.g., dexamethasone), one or more other compounds such as sodium thiosulfate, one or more other compounds such as gacyclidine, one or more other factors such as brain derived neurotropic factor (BDNF), one or more other factors such as gamma-secretase inhibitors, and any combination thereof.

Because human mesenchymal stem cells (MSCs) appear to require epidermal growth factor (EGF) and retinoic acid in culture for their directed differentiated into inner ear sensory cells, in embodiments having PBMT combined with one or more stem cell therapies, the treatment may also comprise use of one or more of epidermal growth factor (EGF), retinoic acid, and any combination thereof. In this manner, the therapy may facilitate differentiation of one or more MSCs into inner ear sensory cells for therapeutic benefit.

In embodiments, PBMT may be combined with the use of light, electricity, and/or acoustical energies in differing patterns and dosing methods to mitigate tinnitus and/or ear ringing and/or to modify the phantom signal generated by the brain which causes tinnitus and/or ear ringing. In embodiments, a treatment for tinnitus and/or ear ringing comprises administration of PBMT, optionally combined with one or more other stimulation therapies, such as electromagnetic, electrical, acoustic, or any combination thereof, wherein a signal of one or more of the PBMT, the electrical, photonic and/or acoustic therapies are adapted and/or changed over time to sustain an optimal safe and effective treatment of tinnitus and/or ear ringing. In embodiments, PBMT alone or in combination with one or more other therapies, stimulates a neurological response in the subject, which may comprise signal creation, neural remodeling, or any combination thereof to impart a therapeutic benefit to the subject.

Generally, PBMT of the present invention may be combined with any established, experimental, or alternative therapies that may be local or systemic in nature. In embodiments, one or more adjunctive therapies may be combined with PBMT of the present invention. Such adjunctive therapies may include, but may not necessarily be limited to, treatment with one or more lipoflavinoids, treatment with ketamine, treatment with MDMA, treatment with LSD, treatment with psilocybin, treatment with hypnosis, treatment with acupuncture, treatment with ginkgo biloba, treatment with a B complex, and any combination thereof. In this manner, the PBMT of the present invention may be enhanced and/or complementary with respect to an adjunctive therapy, an established therapy, an experimental therapy, and/or an alternative therapy.

Variations of the invention—Variations of the invention are included within the scope of this invention and disclosure. Exemplary variations of the invention include, but are not limited to, the inclusion of one or more wavelengths of therapeutic light in the treatment method, which includes narrow band wavelengths, wide band wavelengths, or any combination thereof. While it may be beneficial to utilize primarily red and/or near-IR light, other wavelengths may also provide a therapeutic benefit, including but not necessarily limited to blue light, UV-A light, UV-B light, amber light, blue light, and green light. In embodiments, the photon source device may be configured to emit light having one or more wavelengths including, but not necessarily limited to: 447 nm, 532 nm, 635 nm, 808 nm, and any combination thereof.

In embodiments, the power source may be integral with the photon source device or non-integral with the photon source device, as a battery such as a rechargeable battery or as an external power source such as a larger battery or an alternating current (AC) or direct current (DC) external power source, or any combination thereof. The power of the therapeutic light may be adjustable such that irradiance and radiance, as well as the wavelength and/or wavelengths which are delivered to the portion of the subject receiving therapy, may be selectively adjustable to adjust and/or control the PBMT dosing provided. The photonic illumination plane may be selectively adjustable, or at a defined distance from the light source to the portion of the subject receiving PBMT, or any combination thereof. In addition, the photonic output may include biphasic pulses, monophasic pulses, multiphasic pulses, and any combination thereof. In embodiments, a single photonic source wavelength is provided by the light source, and this is converted into one or more different wavelengths through the use of one or more optics, one or more filters, one or more waveguides, one or more quantum dots (QD's), and any combination thereof. In an embodiment of the system it may be configured to prevent hearing injury from ototoxic drug and/or chemical exposure, age related degeneration, acoustical injury, viral/bacterial infection or any combination thereof. A preventative application could require the subject to be exposed to the PBMT for a period of time prior to exposure to such sources of hearing injury. This preventative treatment may utilize a specific wavelength of light, e.g. 808, 830, 650 nm, that has demonstrated protective capabilities for the subject, ambient environment and injury source.

In embodiments, the system provides local control system and data management, remote control system and data management, or a combination of all, as well as analytic features to determine the subject's therapy progress and adjust therapy during use of the invention. The invention may be configured to track patient compliance, status, and progress, and allow therapy variables to be adjusted remotely, for example, by a remotely located clinician. The system may upload sensor and therapy compliance and device status data to a remote data management and analysis system. In embodiments, sensor and treatment data that pertains to the invention may be reviewed remotely by the subject and/or authorized third parties, e.g., a clinician., In embodiments, the photon source device, the control system, and any combination thereof may include one or more wireless communication interfaces, which enables wireless control of one or more components of thesystem. In embodiments, the control system may upload data to a mobile device such as a smartphone, to a networked data management system, to a cloud-based data management and analytics system, or to another authorized data management system (e.g., such as a system containing protected health information, medical records, or employee records) or any combination thereof. In embodiments, the invention allows analysis and visual display of such results using a mobile device, a networked, cloud, and/or another data management system.

In embodiments, the invention provides a speculum that can be flexible, curved or straight, with a waveguide feature to illuminate the selected tissue region of the subject to deliver optimal safe and effective therapeutic light. The flexible and/or curved speculum may be utilized to mitigate ear canal topology for effective PBMT illumination of the targeted tissue region e.g. .to the middle ear, cochlea and/or inner ear, for optimal hearing loss protection and/or restoration. In embodiments, the invention provides a standard or customized speculum cover that may be disposable or durable. In embodiments, the invention provides a speculum with a protective cover to protect the photon light source from foreign materials from impairing the operation of the device and may enable the adjustment of the optimal safe and effective light illumination to the selected site on the subject. This cover, which may be fixed or adjustable, may be configured with a known impact on the delivery of the photonic output and illumination site on the subject. Alternatively, or in addition, the invention provides one or more algorithms that adjust the system to limit the impact of the cover on the performance of the system. In embodiments, the invention may provide one or more algorithms for the system to create optimal safe and effective light signal that illuminates the selected tissue region of the subject. e.g. inner ear through the tympanic membrane, by adjusting one or more of the following device variables, e.g. irradiance, radiance, time of exposure, sequence, light wavelength, treatment frequency, distance to light source, state of the subject, light modulation, light coherence, site exposure, tissue type, prior treatments, etc. In embodiments, the invention provides a pre-treatment of the selected subject's site with exogenous materials or processes that improve the delivery of the optimal safe and effective therapeutic light signal to the selected site on the subject. Such pre-treatments may comprise the application of one or more reflective substances, materials or devices to the ear canal or within the oral cavity, and in this manner, the optimal safe and effective therapeutic light is able to travel to the selected treatment site with a known amount of signal loss/attenuation through absorption and reflectance into areas of the body adjacent and/or near the treatment site.

In embodiments, the photonic energy delivered to the portion of the subject's body may upregulate and/or stimulate beneficial physiological changes that are associated with, correlated with, and/or causative to mitigate sensorineural auditory acuity (frequency and/or intensity) loss, and/or tinnitus and/or ear ringing. In embodiments, the photonic energy delivered to the selected portion of the subject's body may down regulate and/or inhibit detrimental physiological changes that are associated with, correlated with, and/or causative of sensorineural auditory acuity (frequency and/or intensity) loss, and/or tinnitus and/or ear ringing. In embodiments, the photonic energy delivered to the portion of the subject's body may upregulate and/or stimulate beneficial physiological changes that are associated with, correlated with, and/or causative to mitigate sensorineural auditory acuity (frequency and/or intensity) loss, and/or tinnitus and/or ear ringing, and also down regulate and/or inhibit detrimental physiological changes that are associated with, correlated with, and/or causative of sensorineural auditory acuity (frequency and/or intensity) loss, and/or tinnitus and/or ear ringing.

In embodiments, the invention may utilize one or more waveguides (may be flexible, curved or straight) to deliver the therapeutic light to the selected portion of the subject's body. The flexible, straight and/or curved waveguide may be utilized to mitigate the ear canal curvature for effective delivery of PBMT to the middle ear and/or inner ear or any combination thereof. The invention may provide fine-tuned control over duty cycle, light wavelength, treatment frequency, sequence, pulse shape, therapy time, minimum to maximum light control, and/or increasing and decreasing power/energy for purposes of stimulating, inhibiting, and/or stimulating and inhibiting one or more biological responses in a single or multiple applications of the PBMT. In embodiments, the invention utilizes a vertical-cavity surface-emitting laser (VCSEL), which is a type of semiconductor laser diode with laser beam emission perpendicular from the top surface, which is contrary to conventional edge-emitting semiconductor lasers (also known as in-plane lasers). In embodiments of the invention, the invention stimulates one or more physiological responses, which may include one or more nerve cells.

In embodiments, the invention provides a mechanism that utilizes an external force (e.g. electromagnetic, e.g. near infrared light, X-ray, ultrasound, and the like) to change the activation state of one or more chemical and/or biological compounds to elicit a therapeutic outcome. In embodiments, the present invention may be utilized for wound healing therapy, for example, after tympanostomy tube insertion and/or removal, cochlear implant, post intratympanic injections and/or after tympanic membrane rupture. In this manner, the wound obtained from any of these procedures or injuries, e.g. tympanostomy tube insertion and/or removal, cochlear implant surgery, may be more effectively healed.

In embodiments, the invention provides systems, devices, and methods of utilizing PBMT to produce one or more biological responses in the subject. In embodiments, PBMT may generate one or more biological responses by varying stimulation sequence, irradiance, treatment time, patterns, duty cycle, sequence, wavelengths, location, and exposure area. In embodiments, PBMT of the present invention may stimulate and/or inhibit the cellular respiratory electron transport chain for optimal treatment based on a state of the subject. In embodiments, PBMT of the present invention may enable cellular REDOX regulation and related ROS and/or oxidative stress for optimal treatment based on a state of the subject. In embodiments, PBMT of the present invention may inhibit and/or stimulate cellular ATP for optimal treatment based on a state of the subject. In embodiments, PBMT of the present invention may manage cellular apoptosis and/or necrosis for optimal treatment based on a state of the subject. In embodiments, PBMT of the present invention may manage cellular nitric oxide (NO), cyclooxygenase (COX), and/or the interfacial water layer (IWL) responses for optimal treatment based on a state of the subject. In embodiments, PBMT of the present invention may upregulate messenger molecules, including but not necessarily limited to ROS and NO, which in turn may activate transcription factors such as NF-κB and AP-1, which may enter the nucleus and cause transcription of a range of new gene products for optimal treatment based on a state of the subject. In embodiments, PBMT of the present invention may mitigate, manage, or both mitigate and manage an underlying biological state of the subject to prevent further degradation of auditory acuity, hearing loss and associated side effects, e.g., tinnitus and/or ear ringing.

In embodiments, the PBMT systems, devices, and methods may deliver an optimal safe and effective light therapy to maintain a beneficial and/or therapeutic quantity of cellular compounds e.g. ATP, NO, ROS, in one or more cells, tissues, and/or biological structures of the subject. In embodiments, the optimal therapeutic light energy may be between 0.5 and 5.0 J/cm² at the selected tissue site on the subject. In embodiments, the optimal therapeutic light energy at one or more selected tissue sites on the subject may be about 2.8 J/cm² or exactly 2.8 J/cm². The amount of therapeutic light energy delivered to one or more selected tissue sites will be based upon the needed physiological response, e.g. stimulation, inhibition or a combination of those responses. An example is therapeutic light may be delivered to a portion of a subject's body for maintenance or optimization of the cellular compounds e.g. ATP, NO, ROS , levels in one or more cells, tissues, or biological structures of the subject. In embodiments, the PBMT systems, devices, and methods may deliver an optimal safe and effective light energy to maximize the amount of cellular compounds, e.g. ATP, NO, ROS in one or more cells, tissues, and/or biological structures of the subject, by varying the time of treatment. In embodiments, the time of treatment may be between 15 and 30 minutes, depending on the amount of the cellular compounds , e.g. ATP, NO, ROS, that is desired to be produced from the PBMT stimulation.

In embodiments, the PBMT systems, devices, and methods may utilize one or more light wavelengths including, but not necessarily limited to: 447 nm, 532 nm, 635 nm, 808 nm, and any combination thereof. In embodiments, the one or more light wavelengths utilized may comprise one or more additional light wavelengths which may be adjacent to the utilized light wavelength. For example, to deliver a nominal wavelength, a range of wavelengths around the nominal wavelength may be included in the PBMT. As a non-limiting example, to deliver 447 nm light to a subject for PBMT, a range of wavelengths may be delivered, wherein 447 nm is within the range, e.g., 446 nm to 448 nm, 445 nm to 449 nm, and 444 nm to 450 nm. The range of wavelengths may be expressed as a nominal wavelength plus or minus a surrounding range of wavelengths, or may be expressed as a percentage of the nominal wavelength, or may be expressed as a range having a minimum and a maximum, as would be understood by a person having ordinary skill in the art. As a non-limiting example, to deliver 447 nm light to the subject for PBMT, light represented as 447±1 nm may be delivered. Similarly, to deliver 447 nm light to the subject for PBMT, light represented as 447±2 nm may be delivered.

In embodiments, therapeutic light of the PBMT of the present invention may comprise, may consist essentially of, or may consist of light with a nominal wavelength of 447 nm. In embodiments, the light may have a wavelength of 447±1 nm, 447±2 nm, 447 ±3 nm, 447±4 nm, 447±5 nm, or 447±10 nm or more. In this manner, in a sense, any range of wavelengths of light which includes 447 nm may be utilized in embodiments.

In embodiments, therapeutic light of the PBMT of the present invention may comprise, may consist essentially of, or may consist of light with a nominal wavelength of 532 nm. In embodiments, the light may have a wavelength of 532±1 nm, 532±2 nm, 532 ±3 nm, 532±4 nm, 532±5 nm, or 532±10 nm or more. In this manner, in a sense, any range of wavelengths of light which includes 532 nm may be utilized in embodiments.

In embodiments, therapeutic light of the PBMT of the present invention may comprise, may consist essentially of, or may consist of light with a nominal wavelength of 635 nm. In embodiments, the light may have a wavelength of 635±1 nm, 635±2 nm, 635 ±3 nm, 635±4 nm, 635 35 5 nm, or 635±10 nm or more. In this manner, in a sense, any range of wavelengths of light which includes 635 nm may be utilized in embodiments.

In embodiments, therapeutic light of the PBMT of the present invention may comprise, may consist essentially of, or may consist of light with a nominal wavelength of 808 nm. In embodiments, the light may have a wavelength of 808±1 nm, 808±2 nm, 808 ±3 nm, 808±4 nm, 808±5 nm, or 808±10 nm or more. In this manner, in a sense, any range of wavelengths of light which includes 808 nm may be utilized in embodiments.

In embodiments, the invention provides PBMT that includes two or more light wavelengths which are combined, sequential, overlapping, or any combination thereof. In embodiments, the PBMT includes a combination of all sequences in one or more illumination sequences. In embodiments, the PBMT utilized may depend on a treatment plan and may include one or more of a pre-treatment, a treatment, and/or a post-treatment, or any combination thereof. Each of these treatments may have the same or different purpose, e.g. protection, stimulation, inhibition or any combination thereof. In embodiments, the treatment method may vary one or more of the irradiance, the treatment time, the treatment wavelength, the treatment location, the treatment exposure area, and the treatment stimulation and/or inhibition illumination sequence or any combination thereof.

Implementations

The operations, algorithms, and methods of the present invention may generally be implemented in suitable combinations of software, hardware, firmware, or a combination thereof, and the provided functionality may be grouped into a number of components, modules, and/or mechanisms. Modules can constitute either software modules (e.g., code embodied on a non-transitory machine-readable medium) or hardware-implemented modules. A hardware-implemented module is a tangible unit capable of performing certain operations and can be configured or arranged in a certain manner. In example embodiments, one or more computer systems (e.g., a standalone, client, or server computer system) or one or more processors can be configured by software (e.g., an application or application portion) as a hardware-implemented module that operates to perform certain operations as described herein.

In embodiments, a hardware-implemented module can be implemented mechanically or electronically. For example, a hardware-implemented module can comprise dedicated circuitry or logic that is permanently configured (e.g., as a special-purpose processor, such as a field programmable gate array (FPGA) or an application-specific integrated circuit (ASIC)) to perform certain operations. A hardware-implemented module can also comprise programmable logic or circuitry (e.g., as encompassed within a general-purpose processor or other programmable processor) that is temporarily configured by software to perform certain operations. It will be appreciated that the decision to implement a hardware-implemented module mechanically, in dedicated and permanently configured circuitry, or in temporarily configured circuitry (e.g., configured by software) can be driven by cost and time considerations.

Accordingly, the term “hardware-implemented module” should be understood to encompass a tangible entity, be that an entity that is physically constructed, permanently configured (e.g., hardwired), or temporarily or transitorily configured (e.g., programmed) to operate in a certain manner, to perform certain operations described herein, or both. Considering embodiments in which hardware-implemented modules are temporarily configured (e.g., programmed), each of the hardware-implemented modules need not be configured or instantiated at any one instance in time. For example, where the hardware-implemented modules comprise a general-purpose processor configured using software, the general-purpose processor can be configured as respective different hardware-implemented modules at different times. Software can accordingly configure a processor, for example, to constitute a particular hardware-implemented module at one instance of time and to constitute a different hardware-implemented module at a different instance of time.

Hardware-implemented modules can provide information to, and receive information from, other hardware-implemented modules. Accordingly, the described hardware- implemented modules can be regarded as being communicatively coupled. Where multiple such hardware-implemented modules exist contemporaneously, communications can be achieved through signal transmission (e.g., over appropriate circuits and buses that connect the hardware- implemented modules). In embodiments in which multiple hardware-implememted implemented modules are configured or instantiated at different times, communications between such hardware- implemented modules can be achieved, for example, through the storage and retrieval of information in memory structures to which the multiple hardware-implemented modules have access. For example, one hardware-implemented module can perform an operation and store the output of that operation in a memory device to which it is communicatively coupled. A further hardware-implemented module can then, at a later time, access the memory device to retrieve and process the stored output. Hardware-implemented modules can also initiate communications with input or output devices, and can operate on a resource (e.g., a collection of information).

The various operations of example methods described herein can be performed, at least partially, by one or more processors that are temporarily configured (e.g., by software) or permanently configured to perform the relevant operations. Whether temporarily or permanently configured, such processors can constitute processor-implemented modules that operate to perform one or more operations or functions. The modules referred toherein can, in some example embodiments, comprise processor-implemented modules.

Similarly, the methods described herein can be at least partially processor-implemented. For example, at least some of the operations of a method can be performed by one of processors or processor-implemented modules. The performance of certain of the operations can be distributed among the one or more processors, not only residing within a single machine, but deployed across a number of machines. In embodiments, the processor orprocessors can be located in a single location (e.g., within an office environment, or a server farm), while in other embodiments the processors can be distributed across a number of locations.

The one or more processors can also operate to support performance of the relevant operations in a “cloud computing” environment or as a “software as a service” (SaaS).

For example, at least some of the operations can be performed by a group of computers (as examples of machines including processors), these operations being accessible via a network(e.g., the Internet) and via one or more appropriate interfaces (e.g., application program interfaces (APIs)).

Example embodiments can be implemented in digital electronic circuitry, in computer hardware, firmware, or software, or in combinations thereof. Example embodiments can be implemented using a computer program product, e.g., a computer program tangibly embodied in an information carrier, e.g., in a machine-readable medium for execution by, or to control the operation of, data processing apparatus, e.g., a programmable processor, a computer, or multiple computers.

A computer program can be written in any form of description language, including compiled or interpreted languages, and it can be deployed in any form, including as a standalone program or as a module, subroutine, or other unit suitable for use in a computing environment. A computer program can be deployed to be executed on one computer or on multiple computers at one site or distributed across multiple sites and interconnected by a communication network.

In example embodiments, operations can be performed by one or more programmable processors executing a computer program to perform functions by operating on input data and generating output. Method operations can also be performed by, and apparatus of example embodiments can be implemented as, special purpose logic circuitry, e.g., an FPGA or an ASIC.

The computing system can include clients and servers. A client and server are generally remote from each other and typically interact through a communication network. The relationship of client and server arises by virtue of computer programs running on the respective computers and having a client-server relationship to each other. In embodiments deploying a programmable computing system, it will be appreciated that both hardware and software architectures merit consideration. Specifically, it will be appreciated that the choice of whether to implement certain functionality in permanently configured hardware (e.g., an ASIC),in temporarily configured hardware (e.g., a combination of software and a programmable processor), or a combination of permanently and temporarily configured hardware can be a design choice. Below are set out hardware (e.g., machine) and software architectures that can be deployed, in various example embodiments.

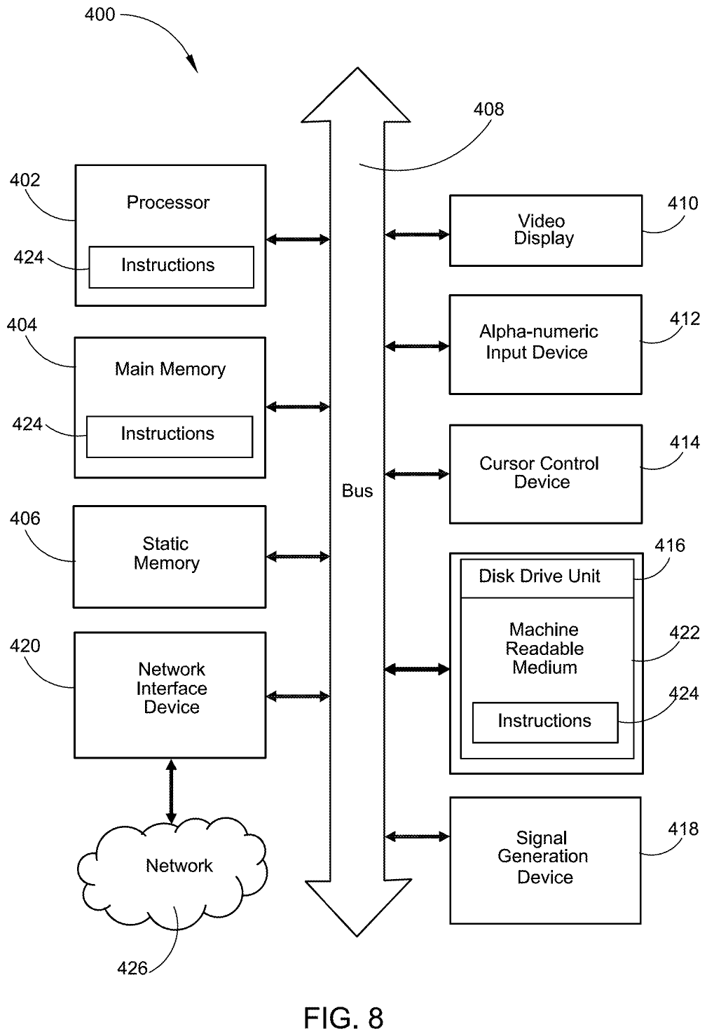

FIG. 8 depicts a schematic block diagram of the photobiomodulation computer system 400 bus architecture illustrating the numerous communications capabilities between the system bus 408 and the hardware elements integrated into the previously described photobiomodulation device 10A-10D.

Referring now to FIG. 8, which depicts a block diagram of a machine in the example form of a computer system 400 within which various instructions 424 may be executed to cause the machine to perform any one or more of the methodologies discussed herein. In alternative embodiments, the machine operates as a standalone device or can be connected (e.g., networked) to other machines. In a networked deployment, the machine can operate in the capacity of a server or a client machine in server-client network environment, or as a peer machine in a peer-to-peer (or distributed) network environment. The machine can be a personal computer (PC), a tablet PC, a set-top box (STB), a personal digital assistant (PDA), a cellular telephone, a web appliance, a network router, switch, or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 400 includes a processor 402 (e.g., a central processing unit (CPU), a graphics processing unit (GPU), or both), a main memory 404, and a static memory 406, which communicate with each other via a bus 408. The computer system 400 can further include a video display 410 (e.g., a liquid crystal display (LCD) or a cathode ray tube (CRT)). The computer system 400 also includes an alpha-numeric input device 412 (e.g., a keyboard or a touch-sensitive display screen), a user interface (UI) navigation (or cursor control) device 414 (e.g., a mouse), a disk drive unit 416, a signal generation device 418 (e.g., a speaker), and a network interface device 420.

The disk drive unit 416 includes a machine-readable medium 422 on which are stored one or more sets of data structures and instructions 424 (e.g., software) embodying or utilized by any one or more of the methodologies or functions described herein. The instructions 424 can also reside, completely or at least partially, within the main memory 404 or within the processor 402, or both, during execution thereof by the computer system 400, with the main memory 404 and the processor 402 also constituting machine-readable media.

While the machine-readable medium 422 is shown in an example embodiment to be a single medium, the term “machine-readable medium” can include a single medium or multiple media (e.g., a centralized or distributed database, and/or associated caches and servers) that store the one or more instructions 424 or data structures. The term “machine- readable medium” shall also be taken to include any tangible medium that is capable of storing, encoding, or carrying instructions 424 for execution by the machine and that cause the machine to perform any one or more of the methodologies of the present disclosure, or that is capable of storing, encoding, or carrying data structures utilized by or associated with such instructions 424. The term “machine-readable medium” shall accordingly be taken to include, but not be limited to, solid-state memories, and optical and magnetic media. Specific examples of machine- readable media 422 include non-volatile memory, including by way of example semiconductor memory devices, e.g., erasable programmable read-only memory (EPROM), electrically erasable programmable read-only memory (EEPROM), and flash memory devices; magnetic disks such as internal hard disks and removable disks; magneto-optical disks; and CD-ROM and DVD-ROM disks.

The instructions 424 can be transmitted or received over a communication network 426 using a transmission medium. The instructions 424 can be transmitted using the network interface device 420 and any one of a number of well-known transfer protocols (e.g., HTTP). Examples of communication networks include a local area network (LAN), a wide area network (WAN), the Internet, mobile telephone networks, plain old telephone (POTS) networks, and wireless data networks (e.g., Wi-Fi and WiMax networks). The term “transmission medium” shall be taken to include any intangible medium that is capable of storing, encoding, or carrying instructions 424 for execution by the machine, and includes digital or analog communications signals or other intangible media to facilitate communication of such software.

In embodiments, one or more components of the invention (e.g., the photon source device, the control system) may be configured for communication via radiofrequency (RF). In embodiments, the one or more components may send and/or receive data by transfer via RF to enable control of the device by another device. In additional embodiments, one or more components of the invention may be configured for communication via acoustic means, optical means, or both acoustic and optical means, and may include a microphone for receiving audio commands from a subject or individual, and/or may include a photosensor for receiving optical commands from the subject or individual. In embodiments, the one or more components of the invention may be configured for communication via RF, acoustic, and optical means, and in this manner the number of possible ways to control the invention may be increased or improved.

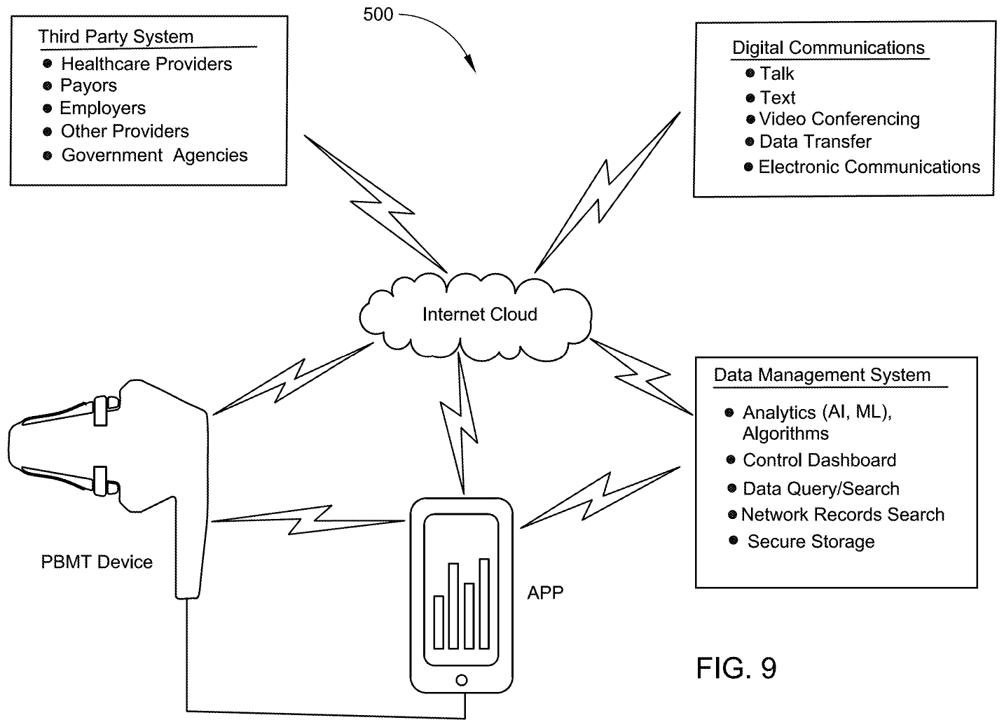

FIG. 9 depicts a schematic diagram of the various telecommunications 500 capabilities of the PBMT device, either alone or coupled to a smartphone utilizing a smartphone application (APP), or other like computing device. As shown, the PBMT device communicates wirelessly or by a connection wire to a smartphone APP and/or wirelessly directly to the internet/cloud. The smartphone/computing device is capable of communications directly to the internet/cloud or a local or remote data management system. Both the PBMT device and the smartphone/computing device communicate wirelessly to perform digital communications (talk, text and video) via the internet/cloud. The PBMT device and the smartphone/computing device communicate wirelessly to third party systems including providers, payors, employers and government agencies.

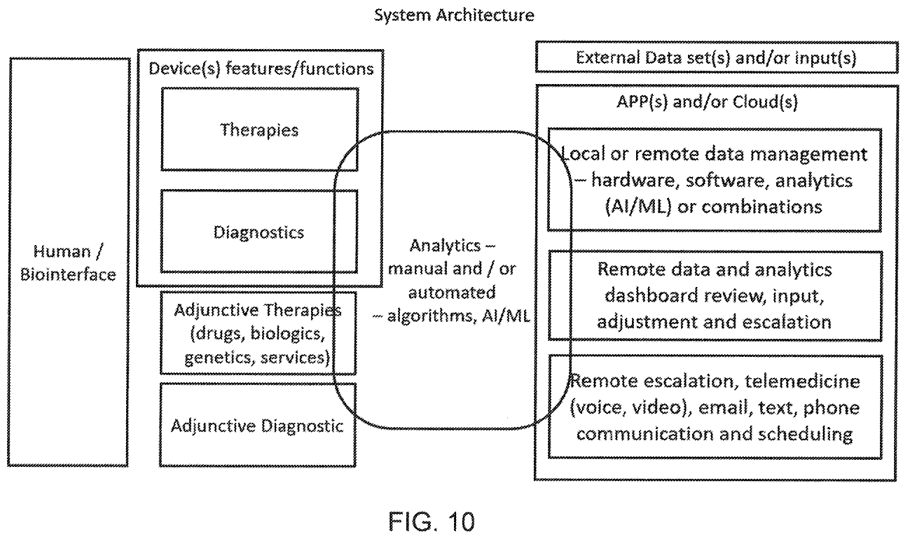

FIG. 10 depicts a flow chart illustrating the system architecture interrelationships between key elements of the system, including the human/biointerface, the therapeutic and diagnostic features and/or functions, adjunctive therapies and/or diagnostics, analytic capabilities, data management system, and the external data sets and inputs. The system's data management, algorithms and analytic capabilities can be located within the device, within a smartphone APP, within a local and/or remote data management system or combinations of each of these elements. The therapeutic and diagnostic algorithms can be updated manually or automatically with data collected from the subject, authorized third parties, devices, analytic system and/or from external sources, e.g. adjunctive therapies, adjunctive diagnostics, electronic health records, manual input by subject or authorized third party. The external data can be manually or automatically imported into the data management system, the smart phone APP, and/or device for use, analysis, storage or a combination of those purposes.

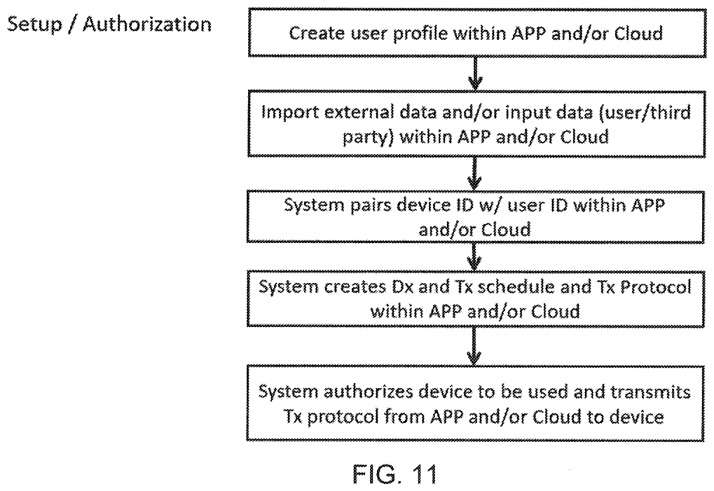

FIG. 11 depicts a flow chart illustrating the typical setup/authorization steps in which a subjects profile can be created by the subject or authorized third party with direct input, importation of external data or a combination of any of those methods. This sequence of steps may manually or automatically pair the assigned device(s), smart phone APP to the subject profile and records. After the creation of the subject profile, pairing of the device(s) and APPs the system may manually or automatically create the initial diagnostic and therapy protocols and transmit such protocols to the devices. The system may then authorize the devices to begin such diagnostic and therapeutic functions as defined in the protocols. The diagnostic and therapeutic protocols may be periodically manually or automatically updated with data from external sources and/or from the devices. The diagnostic and therapeutic protocols can include the scheduled time period in which they are performed.

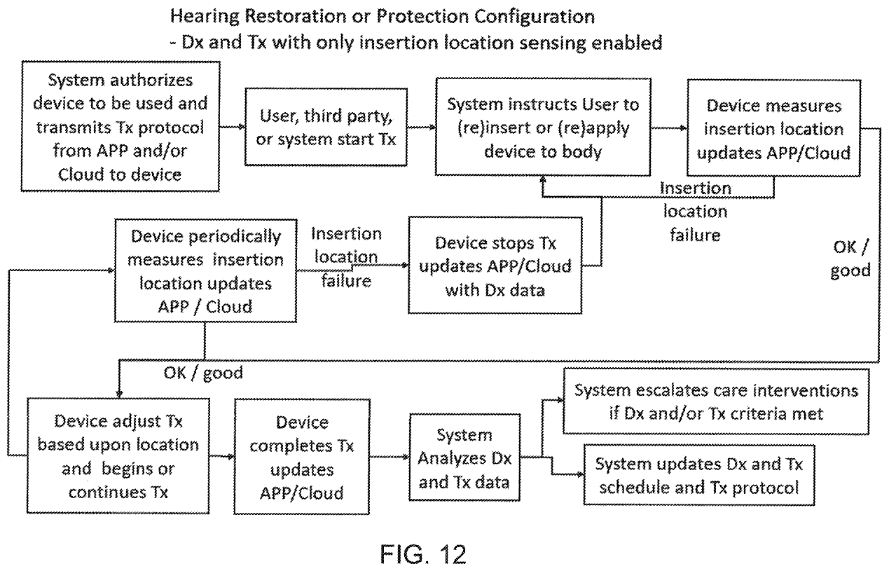

FIG. 12 depicts a flow chart illustrating the use of the system in a hearing restoration and/or protection configuration having the diagnostic and therapeutic functions that are enabled after the system is setup as depicted in FIG. 11 and the devices properly inserted and/or affixed to the subject's body. The detection of proper insertion and/or affixing to the subject's body is determined by a sensing capability within the device and can be performed initially and periodically. The system may require a positive confirmation from the sensing feature that determines proper insertion/affixing to enable the therapeutic energy to be output from the device when it is initially inserted/affixed to subject and/or while in use. These data may be periodically transferred to the APP and/or data management system for analysis, display, storage and/or transfer to separate data management systems. As noted in FIG. 10 these features and functions may be within the device, adjacent APP, data management system or combinations of these configurations. The data management system and/or analytics may utilize the data generated by the system and/or external data imported during use to update the diagnostic and therapeutic protocols manually or automatically. The data management system and/or APP may enable review of the system data by the subject or authorized third parties, including but not limited to protocols, measured diagnostic sensing, and/or analyzed data sets. The data management system may enable alerts and/or notifications to the subject and/or authorized third parties to review data from one or more subjects and/or escalate care. Escalation of care can include manual or automated scheduling of appointments, digital communication messaging (text, phone calls, video calls), procedures, and adjunctive testing.

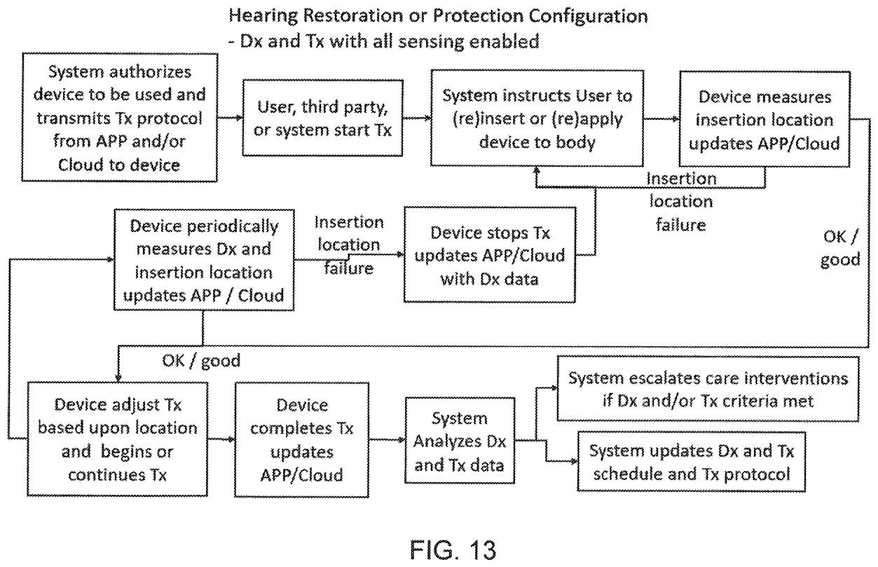

FIG. 13 depicts a flow chart illustrating the use of the system in a hearing restoration and/or protection configuration having the diagnostic and therapeutic functions that are enabled after the system is setup as depicted in FIG. 11 and the devices properly inserted and/or affixed to the subject's body. The detection of proper insertion and/or affixing to the subject's body is determined by a sensing capability within the device and can be performed initially and periodically. The system may require a positive confirmation from the sensing feature that determines proper insertion/affixing to enable the therapeutic energy to be output from the device when it is initially inserted/affixed to subject and/or while in use. The devices may periodically and/or continuously measure the subject's physiological bioparamenters and/or device performance/status with integrated sensing capabilities. These data may be periodically transferred to the APP and/or data management system for analysis, display, storage and/or transfer to separate data management systems. As noted in FIG. 10 these features and functions may be within the device, adjacent APP, data management system or combinations of these configurations. The data management system and/or analytics may utilize the data generated by the system and/or external data imported during use to update the diagnostic and therapeutic protocols manually or automatically. The data management system and/or APP may enable review by the subject or authorized third parties of the system data, including but not limited to protocols, measured diagnostic sensing, and analyzed data sets. The data management system may enable alerts and/or notifications to authorized third parties to review data from one or more subjects and/or escalate care. Escalation of care can include manual or automated scheduling of appointments, digital communication messaging (text, phone calls, video calls) procedures, and adjunctive testing.

The various embodiments of the Systems and Methods for Photobiomodulation primary elements will include as prominent configurations, design and operational functions:

Element 1—one or more light sources which are therapeutic energy adjusted for location on the subject for optimal therapy results.

Element 2—one or more light sources which are therapeutic energy adjusted from previously performed diagnostic test results data for optimal therapy results.

Element 3—one or more light sources in which therapeutic energy is adjusted when device location changes on the body during therapy.

Element 4—elements 1-3 above in varying combinations.

Element 5—elements 1-4 above light sources wavelengths are adjusted for optimal therapy results.

Element 6—elements 1-4 above wherein the light sources energy output is adjusted for optimal therapy results.

Element 7—elements 1-4 above wherein the area of body illuminated by light energy is adjusted for optimal therapy results.

Element 8—elements 5-7 above in varying combinations.

Element 9—elements 1-8 above with one or more of following diagnostic capabilities:

(a) Auditory Tests: evoke potential auditory brainstem response (ABR) and/or auditory steady-state response - ASSR, otoacoustic emissions (OAE), Pure-Tone, Speech Testing, Word tests e.g. Words in Noise, Digits in Noise, tests of the middle ear;

(b) Physiological: Temperature e.g. ear, skin, tissue, core, skin color, skin topology, tissue bioimpedance, galvanic skin response/skin conductance, electroencephalogram—EEG, evoked potential voltages, heart rate, heart rate variability, electro cardiogram, SpO2, StO2, blood pressure, pulse wave velocity, blood flow, respiration rate, respiratory volume, respiratory noise, VO₂ max, tissue composition, motion, body position, ambient noise, otitis media, cerumen, optical and/or acoustic ear canal and tympanic membrane topography scans, 2D and/or 3D images and/or models, algorithmically transformations of one or more of these physiological parameters into a different bioparameter, and/or other electrical, optical or mechanical physiological measurements.

Element 10—elements 1-9 above with an advanced analytics capabilities system and/or device generated diagnostics and/or therapy data.

Element 11—elements 1-10 above with an advanced analytics capabilities system and/or device generated diagnostic and/or therapy data, and/or externally input data, and/or imported external data.

Element 12—elements 1-11 above analytic data output that adjusts diagnostic and therapeutic schedules based on prior analyzed data sets from subject and/or other subjects.

Element 13—elements 1-12 above analytic data output that adjusts therapeutic PBMT protocols based on prior analyzed data sets from subject and/or other subjects.

Element 14—elements 1-13 above data management system generated data for review by subject and/or authorized third party.

Element 15—elements 1-14 above combined with one or more other therapies such as:

(a) Exogenous chemicals e.g. pharmaceutical drugs, biologics, gene therapies e.g. stem cells, supplements;

(b) Devices—hearing aids, sound amplification; noise protection, communication devices, therapeutic devices;

(c) Services—Acupuncture, surgery, meditation, auditory training, brain plasticity remodeling training.

Element 16—elements 1-15 above fully integrated into one or more devices on the body—ear pod, headphone, noise protection, hearing-aid, personal sound amplification, communication devices.

Element 17—elements 1-15 above with system features and functions located on an on-body device and one or more adjacent computing devices, e.g. smartphone, computer, tablet or similar.

Element 18—elements 1-15 above with system features and functions located on an on-body device, and one or more adjacent computing devices, and one or more remote data management and analytic systems.

Element 19—elements 1-18 above with one or more data management and analytic systems that manually or automatically escalate subject care interventions utilizing data from current and/or prior diagnostic and therapy data analysis by one or more of the system analytic features. These interventions can be one or more of the following: Send one or more electronic/digital communication notifications (text, email, voicemail, etc.) to one or more authorized third parties for review and/or action; Automatically create a notification to review analyzed and historical data within data management system by one or more authorized third parties; Automatically scheduling an appointment and/or meeting with subject and authorized third party either in person or through other electronic/digital means, e.g. telemedicine, virtual presence, telephonic or televideo.

Element 20—elements 1-19 above with automated methods and features to enable manual or automated payment invoicing to authorized third parties for services provided, subscriptions and/or other goods and services, e.g. insurance, health savings accounts, credit/debit cards, employers, government agencies, individual service providers, etc.

Element 21—elements 1-19- above with automated methods and procedures to transfer data created, analyzed, imported and/or stored within data management system to authorized third parties.

In summary then, this application relates to systems, devices, and methods for diagnosing, preventing, and treating diseases and disorders through photobiomodulation therapy, either alone or in combination with one or more other therapies. More particularly, the present invention provides photon source devices configured to deliver light to a portion of an organism, which causes a physiological response within that light exposed organism. The invention also provides a system which includes one or more photon source devices and functionality for diagnosing or assessing a disease or disorder, and for monitoring responsiveness of the disease or disorder to treatment with the therapeutic light. Additionally, this application is directed to utilizing the present systems and devices in combination with known adjunctive therapies including devices, services, drugs, biologics, genetics and supplements to produce synergistic optimal therapeutic outcomes.

With respect to the above description then, it is to be realized that the optimum dimensional relationships for the parts of the Systems and Methods for Photobiomodulation, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present design. Therefore, the foregoing is considered as illustrative only of the principles of the Systems and Methods for Photobiomodulation. Further, since numerous modifications and changes will readily occur to those skilled in the art, it is not desired to limit the Systems and Methods for Photobiomodulation to the exact construction and operation shown and described, and accordingly, all suitable modifications and equivalents may be resorted to falling within the scope of this application.

The Systems and Methods for Photobiomodulation 10A, 10B, 10C, 10D, 100, 200 and 300 shown in the drawings and described in detail herein disclose arrangements of elements of particular construction and configuration for illustrating preferred embodiments of structure and method of operation of the present application. It is to be understood, however, that elements of different construction and configuration and other arrangements thereof, other than those illustrated and described may be employed for providing the Systems and Methods for Photobiomodulation 10A, 10B, 10C, 10D, 10, 200 and 300 in accordance with the spirit of this disclosure, and such changes, alternations and modifications as would occur to those skilled in the art are considered to be within the scope of this design as broadly defined in the appended claims.

While certain embodiments of the inventions have been described, these embodiments have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. For example, one portion of one of the embodiments described herein can be substituted for another portion in another embodiment described herein. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification including any accompanying claims, abstract and drawings, and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification including any accompanying claims, abstract and drawings, or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without a subject input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

Further, the purpose of the foregoing abstract is to enable the U.S. Patent and Trademark Office, foreign patent offices worldwide and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

We claim:

1. A photobiomodulation system photon source device, comprising:
(a) a housing having a fitted bio-interface and an interior section having a photon aperture, said housing including one or more adjustable light sources located on and within said housing, wherein said one or more adjustable light sources are capable of emitting photonic energy;
(b) the photobiomodulation system photon source device, wherein said one or more adjustable light sources further includes a waveguide, and a light energy modulator;
(c) a control system in communication with said one or more adjustable light sources; and
(d) a power source in communication with said control system;
wherein when said housing having a fitted bio-interface is placed on or near a subject said one or more adjustable light sources are adjusted to deliver photonic energy emissions to a subject's tissues for therapeutic and diagnostic purposes.

2. The photobiomodulation system according to claim 1, wherein a placement detection system comprised of one or more placement sensors located on or within device wherein said one or more placement sensors are capable of detecting the positioning of said device on or near the subject, and as a result said one or more adjustable light sources are adjusted to deliver photonic energy emissions to a subject's tissues for therapeutic and diagnostic purposes

3. The photobiomodulation system photon source device according to claim 1, wherein said control system and said power source are located on and within said housing.

4. The photobiomodulation system photon source device according to claim 1, wherein said housing further includes a proximity sensor, a speaker, and a microphone.

5. The photobiomodulation system photon source device according to claim 1, wherein said housing having a fitted bio-interface is configured to be placed on and within a subject's ear.

6. The photobiomodulation system photon source device according to claim 5, wherein one or more said housing are configured in a pair to be placed on and within each of a subject's ears.

7. The photobiomodulation system photon source device according to claim 6, wherein said one or more said housing are configured in a pair to be placed on and within each of a subject's ears are integrated into a headset unit.

8. The photobiomodulation system photon source device according to claim 1, wherein said control system and said power source are configured within a smartphone and said control system and one or more light sources are in communication with said smartphone and controlled using a smartphone application, and further wherein said smartphone application is in communication with cloud-based information sources.

9. The photobiomodulation system photon source device according to claim 8, wherein said one or more light sources are located external to said housing and are in communication with said control system via a smartphone application.

10. The photobiomodulation system photon source device according to claim 1, wherein said power source is external to said housing and is in communication with said control unit via a control wire.

11. A method for making a photobiomodulation system photon source device, comprising the steps of:
(a) providing a housing having a fitted bio-interface and an interior section having a photon aperture, said housing including one or more adjustable light sources located on and within said housing, wherein said one or more light sources are capable of emitting photonic energy;
(b) providing said one or more adjustable light sources wherein said one or more adjustable light sources further includes a waveguide and a light energy modulator;
(c) providing a control system in communication with said one or more light sources; and
(d) providing a power source in communication with said control system;
wherein when said housing having a fitted bio-interface is placed on or near a subject said one or more adjustable light sources are adjusted to deliver photonic energy emissions to a subject's tissues for therapeutic and diagnostic purposes.

12. The method for making a photobiomodulation system photon source device according to claim 11, wherein said control system and said power source are located on and within said housing.

13. The method for making a photobiomodulation system photon source device according to claim 11, wherein said one or more adjustable light sources further includes a waveguide, a light energy modulator, a light source aimer, and a light collimating feature.

14. The method for making a photobiomodulation system photon source device according to claim 11, wherein said housing further includes a proximity sensor, a speaker, and a microphone.

15. The method for making a photobiomodulation system photon source device according to claim 11, wherein said housing having a fitted bio-interface is configured to be placed on and within a subject's ear.

16. The method for making a photobiomodulation system photon source device according to claim 15, wherein one or more said housing are configured in a pair to be placed on and within each of a subject's ears.

17. The method for making a photobiomodulation system photon source device according to claim 16, wherein said one or more said housing are configured in a pair to be placed on and within each of a subject's ears are integrated into a headset unit.

18. The method for making a photobiomodulation system photon source device according to claim 11, wherein said control system and said power source are configured within a smartphone and said control system and one or more light sources are in communication with said smartphone and directed using a smartphone application, and further wherein said smartphone application is in communication with cloud-based information sources.

19. The method for making a photobiomodulation system photon source device according to claim 18, wherein said one or more light sources are located external to said housing and are in communication with said control system via a smartphone application.

20. A method for using a photobiomodulation system photon source device, comprising the steps of:
(a) setting up and authorizing a photobiomodulation system photon source device by wireless communications thereby creating a subject profile and importing external data and subject and third party input data;
(b) pairing a system device identifier with a subject identification using a smartphone application and a cloud-based information database;
(c) creating system subject diagnostic schedules and protocols based on said subject data via said smartphone application and a cloud-based information database;
(d) creating system subject therapeutic schedules and protocols based on said subject data via said smartphone application and a cloud-based information database;
(e) placing and positioning said photobiomodulation system photon source device using a fitted bio-interface and one or more placement sensors;
(f) performing a baseline diagnostic test for hearing acuity;
(g) activating adjusted photon emissions from one or more light sources and delivering a quantity of light energy to a subject's tissues; and
(h) measuring the efficacy of the delivered light;
wherein the method of using said photobiomodulation system photon source device enables development of optimally customized protocols for preventative therapy treatments by evaluating changes in the physiological state of a subject following the delivery of light energy.
